(19) United States
(12) Patent Application Publication
(10) Pub. No.: US 2024/0042098 A1
(43) Pub. Date: Feb. 8, 2024

(54) Design and Production of Customizable and Highly Aligned Fibrillar Collagen Scaffolds

(71) Applicant: Northeastern University, Boston, MA (US)

(72) Inventors: Cassandra Leigh Martin, Boston, MA (US); Leila Deravi, Cambridge, MA (US); Jeffrey Paten, Mansfield, MA (US)

(21) Appl. No.: 18/264,536

(22) PCT Filed: Mar. 4, 2022
(86) PCT No.: PCT/US2022/070977

§ 371 Date: Aug. 7, 2023

Related U.S. Application Data
(60) Provisional application No. 63/157,605, filed on Mar. 5, 2021.

(51) Int. Cl. A61L 27/24; A61L 27/50; A61L 27/22

(52) U.S. Cl. CPC A61L 27/24; A61L 27/50; A61L 27/222; A61L 27/225; A61L 2400/12

(57) ABSTRACT

Provided herein are methods for preparing fibrillar polymeric scaffolds, compositions comprising fibrillar polymeric scaffold, and an apparatus comprising two concentric cylinders, a rotating inner cylinder powered by a motor, and a stationary outer cylinder.

### RELATED APPLICATION

This application claims the benefit of U.S. Provisional Application No. 63/157,605, filed on Mar. 5, 2021. The entire teachings of this application are incorporated herein by reference.

### BACKGROUND

The extracellular matrix (ECM) is a complex, multi-component network that provides the mechanical strength, instructional cues, and underlying structure of tissues and organs in the body (Frantz, C.; Stewart, K. M.; Weaver, V. M., The extracellular matrix at a glance. J. Cell. Sci. 2010, 123 (Pt 24), 4195-200. DOI: 10.1242/jcs.023820; Hynes, R. O., The extracellular matrix: not just pretty fibrils. Science 2009, 326 (5957), 1216-9. DOI: 10.1126/science.1176009). These networks are customized for region-specific architectures, where fiber diameter and organization directly influence tissue form and function. For example, the human Achilles tendon contains uniaxial fibrils made of type I collagen (COL) that resist failure under load, withstanding forces and stresses up to ˜5000 N and ˜80 MP a, respectively (Cowin, S. C., How is a Tissue Built? J. Biomech. Eng. 2000, 122, 553-569; O'Brien, M., Structure and metabolism of tendons. Scand. J. Med. Sci. Sports 1997, 7 (2), 55-61. DOI: 10.1111/j.1600-0838.1997.tb00119.x; Wren, T. A.; Yerby, S. A.; Beaupre, G. S.; Carter, D. R., Mechanical properties of the human achilles tendon. Clin. Biomech. (Bristol, Avon) 2001, 16 (3), 245-51. DOI: 10.1016/s0268-0033(00)00089-9). On the other hand, the cornea, also comprised of type I COL fibrils but organized as perpendicular sheets, withstands the intraocular pressure in the eye (˜1900 Pa) while maintaining optical transparency (Boote, C.; Dennis, S.; Huang, Y.; Quantock, A. J.; Meek, K. M., Lamellar orientation in human cornea in relation to mechanical properties. J. Struct. Biol. 2005, 149 (1), 1-6. DOI: 10.1016/j.jsb.2004.08.009; Holmes, D. F.; Gilpin, C. J.; Baldock, C.; Ziese, U.; Koster, A. J.; Kadler, K. E., Corneal collagen fibril structure in three dimensions: Structural insights into fibril assembly, mechanical properties, and tissue organization. Proc. Natl. Acad. Sci. U.S.A. 2001, 98 (13), 7307-12. DOI: 10.1073/pnas.111150598; Hamilton, K. E.; Pye, D. C., Young's modulus in normal corneas and the effect on applanation tonometry. Optom. Vis. Sci. 2008, 85 (6), 445-50. DOI: 10.1097/OPX.0b013e3181783a70; Komai, Y.; Ushiki, T., The three-dimensional organization of collagen fibrils in the human cornea and sclera. Invest. Ophthalmol. Vis. Sci. 1991, 32 (8), 2244-58). In an even different manner, the type I COL fibrils in the skin are organized as a woven network that enables the fibrils to straighten, reorient, and slide past each other in order to resist tearing and to redistribute external forces (Crichton, M. L.; Chen, X.; Huang, H.; Kendall, M. A., Elastic modulus and viscoelastic properties of full thickness skin characterised at micro scales. Biomaterials 2013, 34 (8), 2087-97. DOI: 10.1016/j.biomaterials.2012.11.035; Yang, W.; Sherman, V. R.; Gludovatz, B.; Schaible, E.; Stewart, P.; Ritchie, R. O.; Meyers, M. A., On the tear resistance of skin. Nat. Commun. 2015, 6, 6649. DOI: 10.1038/ncomms7649). Given the range of unique structure-dependent functions of precisely aligned COL fibrils in vivo, efforts have been made to recapitulate these features in vitro. Applications of blended ECM-based proteins and proteoglycans, such as Matrigel™, separated proteins and proteoglycans, as well as combinations of these biomolecules with COL, have demonstrated the broad appeal of protein-based fibrous materials in engineered tissue (Kleinman, H. K.; Martin, G. R., Matrigel: Basement membrane matrix with biological activity. Semin. Cancer Biol. 2005, 15 (5), 378-386. DOI: 10.1016/j.semcancer.2005.05.004; Nerger, B. A.; Brun, P.-T.; Nelson, C. M., Microextrusion printing cell-laden networks of type I collagen with patterned fiber alignment and geometry. Soft Matter 2019, 15 (5728); Ghasemi-Mobarakeh, L.; Prabhakaran, M. P.; Morshed, M.; Nasr-Esfahani, M. H.; Ramakrishna, S., Bio-functionalized PCL nanofibrous scaffolds for nerve tissue engineering. Mater. Sci. Eng. C-Mater. 2010, 30 (8), 1129-1136. DOI: 10.1016/j.msec.2010.06.004; Ejim, O. S.; Blunn, G. W.; Brown, R. A., Production of artifical-oriented mats and strands from plasma fibronectin: a morphological study. Biomaterials 1993, 14; Chantre, C. O.; Campbell, P. H.; Golecki, H. M.; Buganza, A. T.; Capulli, A. K.; Deravi, L. F.; Dauth, S.; Sheehy, S. P.; Paten, J. A.; Gledhill, K.; Doucet, Y. S.; Abaci, H. E.; Ahn, S.; Pope, B. D.; Ruberti, J. W.; Hoerstrup, S. P.; Christiano, A. M.; Parker, K. K., Production-scale fibronectin nanofibers promote wound closure and tissue repair in a dermal mouse model. Biomaterials 2018, 166, 96-108. DOI: 10.1016/j.biomaterials.2018.03.006; Chantre, C. O.; Gonzalez, G. M.; Ahn, S.; Cera, L.; Campbell, P. H.; Hoerstrup, S. P.; Parker, K. K., Porous Biomimetic Hyaluronic Acid and Extracellular Matrix Protein Nanofiber Scaffolds for Accelerated Cutaneous Tissue Repair. ACS Appl. Mater. Interfaces 2019, 11 (49), 45498-45510. DOI: 10.1021/acsami.9b17322; Buttafoco, L.; Kolkman, N. G.; Engbers-Buijtenhuijs, P.; Poot, A. A.; Dijkstra, P. J.; Vermes, I.; Feijen, J., Electrospinning of collagen and elastin for tissue engineering applications. Biomaterials 2006, 27 (5), 724-734. DOI: 10.1016/j.biomaterials.2005.06.024; Paten, J. A.; Martin, C. L.; Wanis, J. T.; Siadat, S. M.; Figueroa-Navedo, A. M.; Ruberti, J. W.; Deravi, L. F., Molecular Interactions between Collagen and Fibronectin: A Reciprocal Relationship that Regulates De Novo Fibrillogenesis. Chem 2019, 5 (8), 2126-2145. DOI: https://doi.org/10.1016/j.chempr.2019.05.011). However, it remains technically challenging to generate and scale pure COL fibers. This may be due in part to the fact that the underlying mechanisms regulating COL fibrillogenesis in vivo, specifically the use of auxiliary biomolecules, complex fluid dynamics, and cell-matrix tension as important contributors of this process, are not well understood (Martin, C. L.; Bergman, M. R.; Deravi, L. F.; Paten, J. A., A Role for Monosaccharides in Nucleation Inhibition and Transport of Collagen. Bioelectricty 2020, 2 (2), 1-12; Paten, J. A.; Siadat, S. M.; Susilo, M. E.; Ismail, E. N.; Stoner, J. L.; Rothstein, J. P.; Ruberti, J. W., Flow-Induced Crystallization of Collagen: A Potentially Critical Mechanism in Early Tissue Formation. ACS Nano 2016, 10 (5), 5027-40. DOI: 10.1021/acsnano.5b07756; Holmes, D. F.; Yeung, C. C.; Garva, R.; Zindy, E.; Taylor, S. H.; Lu, Y.; Watson, S.; Kalson, N. S.; Kadler, K. E., Synchronized mechanical oscillations at the cell-matrix interface in the formation of tensile tissue. Proc. Natl. Acad. Sci. U.S.A. 2018, 115 (40), E9288-E9297. DOI: 10.1073/pnas.1801759115).

Despite these challenges, there have been several attempts to generate aligned COL scaffolds. One common approach involves extruding concentrated COL as fibers or films with the aid of external electric fields (electrospinning), centrifugal forces (rotary jet spinning), differential solvent baths (wet spinning), or 3D-printing (Matthews, J. A.; Wnek, G. E.; Simpson, D. G.; Bowlin, G. L., Electrospinning of collagen nanofibers. Biomacromolecules 2002, 3 (2), 232-8. DOI: 10.1021/bm015533u; Zhong, S.; Teo, W. E.; Zhu, X.; Beuerman, R. W.; Ramakrishna, S.; Yung, L. Y., An aligned nanofibrous collagen scaffold by electrospinning and its effects on in vitro fibroblast culture. J. Biomed. Mater. Res. A 2006, 79 (3), 456-63. DOI: 10.1002/jbm.a.30870; Caves, J. M.; Kumar, V. A.; Wen, J.; Cui, W.; Martinez, A.; Apkarian, R.; Coats, J. E.; Berland, K.; Chaikof, E. L., Fibrillogenesis in continuously spun synthetic collagen fiber. J. Biomed. Mater. Res. B Appl. Biomater. 2010, 93 (1), 24-38. DOI: 10.1002/jbm.b.31555; Yaari, A.; Schilt, Y.; Tamburu, C.; Raviv, U.; Shoseyov, O., Wet Spinning and Drawing of Human Recombinant Collagen. ACS Biomater. Sci. Eng. 2016, 2, 349-360). These extrusion-based methods have demonstrated some potential for scalability in materials applications. For instance, Lee et al. illustrated scalability by 3D-printing a 55×37 mm replicate of a neonatal human heart comprised solely of COL (Lee, A.; Hudson, A. R.; Shiwarski, D. J.; Tashman, J. W.; Hinton, T. J.; Yerneni, S.; Bliley, J. M.; Campbell, P. G.; Feinberg, A. W., 3D bioprinting of collagen to rebuild components of the human heart. Science 2019, 365 (6452), 482-487. DOI: In spite of this, it is unclear whether the fibrillar ultrastructure, or arrangement of the monomers, of the COL fibers were retained throughout this process. The most common approach for generating COL fibers at similar scales is electrospinning; however, this technique is met with some controversy in the field, as data suggests that COL is susceptible to denaturation during the spinning process (Ugolis, D. I.; Khew, S. T.; Yew, E. S.; Ekaputra, A. K.; Tong, Y. W.; Yung, L. Y.; Hutmacher, D. W.; Sheppard, C.; Raghunath, M., Electro-spinning of pure collagen nano-fibres - just an expensive way to make gelatin? Biomaterials 2008, 29 (15), 2293-305. DOI: 10.1016/j.biomaterials.2008.02.009; Rogalski, J. J.; Bastiaansen, C. W. M.; Peijs, T., Rotary jet spinning review- a potential high yield future for polymer nanofibers. Nanocomposites 2017, 3 (4), 97-121; Bhardwaj, N.; Kundu, S. C., Electrospinning: a fascinating fiber fabrication technique. Biotechnol. Adv. 2010, 28 (3), 325-47. DOI: 10.1016/j.biotechadv.2010.01.004). When retention of ultrastructure is emphasized, it has often been done so at the cost of scalability. For instance, Saeidi et al. demonstrated that shear flow can align single COL fibrils during the polymerization process in the direction of the flow (Saeidi, N.; Sander, E. A.; Ruberti, J. W., Dynamic shear-influenced collagen self-assembly. Biomaterials 2009, 30 (34), 6581-92. DOI: Other techniques including microfluidics, flow-induced crystallization, differential electrochemistry, magnetic orientation, spin coating, and nanolithography have seen moderate successes in generating and aligning COL fibrils (Lanfer, B.; Freudenberg, U.; Zimmermann, R.; Stamov, D.; Korber, V.; Werner, C., Aligned fibrillar collagen matrices obtained by shear flow deposition. Biomaterials 2008, 29 (28), 3888-95. DOI: 10.1016/j.biomaterials.2008.06.016; Cheng, X.; Gurkan, U. A.; Dehen, C. J.; Tate, M. P.; Hillhouse, H. W.; Simpson, G. J.; Akkus, O., An electrochemical fabrication process for the assembly of anisotropically oriented collagen bundles. Biomaterials 2008, 29 (22), 3278-88. DOI: 10.1016/j.biomaterials.2008.04.028; Torbet, J.; Ronziere, M. C., Magnetic alignment of collagen during self-assembly. Biochem. J. 1984, 219 (3), 1057-9. DOI: 10.1042/bj2191057; Saeidi, N.; Sander, E. A.; Zareian, R.; Ruberti, J. W., Production of highly aligned collagen lamellae by combining shear force and thin film confinement. Acta Biomater. 2011, 7 (6), 2437-47. DOI: 10.1016/j.actbio.2011.02.038; Wilson, D. L.; Martin, R.; Hong, S.; Cronin-Golomb, M.; Mirkin, C. A.; Kaplan, D. L., Surface organization and nanopatterning of collagen by dip-pen nanolithography. Proc. Natl. Acad. Sci. U.S.A. 2001, 98 (24), 13660-4. DOI: 10.1073/pnas.241323198). However, the majority of these procedures lack a realistic pathway to scaling into full tissue replicates and remain limited to producing individual sub-micron diameter fibrils or single, long, micron-sized fibers.

To improve scalability without sacrificing fibrillar ultrastructure, many researchers have focused on re-orienting and aligning pre-assembled fibrils with the aid of magnetic bead-induced alignment, biaxial gel compression, or counter-rotating extrusion (Antman-Passig, M.; Shefi, O., Remote Magnetic Orientation of 3D Collagen Hydrogels for Directed Neuronal Regeneration. Nano Lett. 2016, 16 (4), 2567-73. DOI: Guo, C.; Kaufman, L. J., Flow and magnetic field induced collagen alignment. Biomaterials 2007, 28 (6), 1105-14. DOI: Zitnay, J. L.; Reese, S. P.; Tran, G.; Farhang, N.; Bowles, R. D.; Weiss, J. A., Fabrication of dense anisotropic collagen scaffolds using biaxial compression. Acta Biomater. 2018, 65, 76-87. DOI: 10.1016/j .actbio.2017.11.017; Hoogenkamp, H. R.; Bakker, G. J.; Wolf, L.; Suurs, P.; Dunnewind, B.; Barbut, S.; Friedl, P.; van Kuppevelt, T. H.; Daamen, W. F., Directing collagen fibers using counter-rotating cone extrusion. Acta Biomater. 2015, 12, 113-121. DOI: 10.1016/j.actbio.2014.10.012; Yang, S.; Shi, X.; Li, X.; Wang, J.; Wang, Y.; Luo, Y., Oriented collagen fiber membranes formed through counter-rotating extrusion and their application in tendon regeneration. Biomaterials 2019, 207, 61-75. DOI: 10.1016/j.biomaterials.2019.03.041; Yang, S.; Wang, J.; Wang, Y.; Luo, Y., Key role of collagen fibers orientation in casing-meat adhesion. Food Res. Int. 2016, 89 (Pt 1), 439-447. DOI: 10.1016/j.foodres.2016.08.035). Perhaps the most readily scalable of the three is counter-rotating extrusion, which was originally designed and utilized to prepare synthetic sausage casings but has since been adapted for tissue engineering applications (Harper, B. A.; Barbut, S.; Lim, L.-T.; Marcone, M. F., Microstructural and textural investigation of various manufactured collagen sausage casings. Food Res. 2012, 49, 494-500). This method consists of concentric cones rotating in opposite directions, effectively molding a preformed COL dough into sheets, where the shear from the cones is proposed to pull the COL fibrils into a uniaxial alignment. Yang et al. and Hoogenkamp et al. describe collagenous materials with aligned fibers that persist over centimeter-scale lengths which are at least ten times greater than the other previously mentioned strategies. Lastly, the authors describe the utility of these molded scaffolds in a rat Achilles tendon surgery, illustrating their potential in promoting tenogenic differentiation for tendon repair. Despite these successes, the process of counter-rotating extrusion still requires lengthy pre- and post-processing steps in order to both form and retain the initial material geometry. Furthermore, the longer-term impact of the intensive remodeling and restructuring on the integrity and mechanics of the scaffold is still unknown.

Accordingly, there remains a need for preparing aligned COL scaffolds that is scalable, retains native microstructures, can be done under mild conditions, and does not require the use of preformed COL dough and/or salt precipitation steps.

### SUMMARY

Described herein is a fibrillar polymeric scaffold comprising fibrils derived from one or more polymerizable monomers, wherein the scaffold is non-cellular and about 50% or greater of the fibrils are within ±20° of the orientation of all fibrils in the scaffold.

Also described herein are compositions comprising a (i) fibrillar polymeric scaffold comprising fibrils derived from one or more polymerizable monomers, and (ii) a sacrificial polymerization scaffold, wherein the sacrificial polymerizable scaffold is adhered to the fibrillar polymeric scaffold.

Also described herein are methods for preparing a fibrillar polymeric scaffold comprising: (a) contacting an adherent solid surface with a mixture comprising a polymerizable monomer and an aqueous solvent under conditions suitable to induce adhesion of polymerizable monomer to the adherent solid surface; and (b) applying shear flow to the mixture under conditions suitable to polymerize the polymerizable monomer into fibrils, thereby preparing the fibrillar polymeric scaffold.

Also described herein are methods for preparing a fibrillar polymeric scaffold in an apparatus, wherein the apparatus comprises two concentric cylinders, an outer cylinder that forms a reservoir having a first radius and an inner cylinder having a second radius smaller than the first radius, wherein the outer cylinder and inner cylinder are independently rotatable with respect to one another, and the outer cylinder or inner cylinder comprises an adherent solid surface in contact with the reservoir, the method comprising: (a) providing a mixture comprising a polymerizable monomer and an aqueous solvent in the reservoir under conditions suitable to induce adhesion of polymerizable monomer to the adherent solid surface; and (b) applying shear flow to the mixture under conditions suitable to polymerize the polymerizable monomer into fibrils, thereby preparing the fibrillar polymeric scaffold.

Also described herein are fibrillar polymeric scaffolds produced according to any of the methods for preparing the same described herein.

Also described herein is an apparatus comprising two concentric cylinders, an outer cylinder that forms a reservoir having a first radius, an inner cylinder configured to be rotated by a motor and having a second radius smaller than the first radius, wherein the outer cylinder and inner cylinder are independently rotatable with respect to one another.

The methods and apparatuses for preparing fibrillar polymeric scaffolds described herein can be used to produce fibrillar polymeric scaffolds that are scalable and use simple monomeric precursors. The methods and apparatuses described herein simultaneously polymerize and aligns the monomeric precursor, thus eliminating the need for COL dough. Finally, the methods and apparatuses do not require additional post-processing steps and may be scaled up without diminishing the quality of the scaffolds produced.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows a depiction of a custom-built device comprised of two concentric cylinders. The top cylinder (spindle 102) was connected to a motor and rotated, and the bottom cylinder (protein reservoir 103) could be lowered and raised to engage or disengage the system. Spindle 102 in the embodiment depicted in FIG. 1 was 25.2 mm long, although only approximately 14 mm of spindle 102 were submerged in the reservoir upon engagement of the system, and 10.6 mm in diameter. Reservoir 103 in the embodiment depicted in FIG. 1 is 15.2 mm deep and 12.6 mm in diameter.

FIG. 2A is a cross-sectional view of spinning process, and shows that the spinning process involves multiple steps where monomeric collagen (COL) polymerizes and tethers to the thin, sacrificial layer of gelatin.

FIG. 2B shows an approximately 3.0×1.0 cm scaffold collected from the spindle depicted in FIG. 1 after spinning.

FIG. 2C is a representative scanning electron microscope (SEM) image showing that the resultant fibrils have high alignment and the signature D-banding of fibrillar COL; scale bar=2 μm.

FIG. 3A is a representative SEM image of a five-layered COL scaffold prepared with 2.5 mg/mL COL and spun at 75 s⁻¹. Scale bar=2 μm.

FIG. 3B is a representative SEM image of a rat tail tendon. Scale bar=2 μm.

FIG. 3C is a representative SEM image of a fibrous COL gel that spontaneously polymerized under no shear. Scale bar=2 μm.

FIG. 4A shows the results of fibril alignment analysis determined by measuring the percentage of fibrils that fell within ±20° of the orientation tensor for the underlying image, where n=15 and five measurements were taken on three independent samples. Error is ±standard deviation (SD).

FIG. 4B shows variation of fibril diameter for each structure depicted in FIGS. 3A-C, where n=300 fibrils over three independent samples and error is reported as ±SD. Statistical significance for results shown in FIGS. 4A and 4B are denoted with an asterisk, where p<0.05.

FIG. 5A shows the threshold tethering force value required to align COL chains composed of one molecule in the direction of the flow.

FIG. 5B shows the threshold tethering force value required to align COL chains composed of five molecules in the direction of the flow.

FIG. 5C shows the threshold tethering force value required to align COL chains composed of 10 molecules in the direction of the flow. The results depicted in FIGS. 5A-5C shows that as the chain length increased, the amount of force required to achieve a high P₂ value increased.

FIG. 6A shows the threshold tethering force value required to align COL chains composed of 25 molecules in the direction of the flow.

FIG. 6B shows the threshold tethering force value required to align COL chains composed of 196 molecules in the direction of the flow.

FIG. 6C shows the threshold tethering force value required to align COL chains composed of 625 molecules in the direction of the flow. FIGS. 6A-6C show that when the number of COL monomers increased from one (FIG. 5A) to 25 (FIG. 6A), 196 (FIG. 6B), or 625 (FIG. 6C), the same threshold force value was needed to align all of the molecules in the system.

FIG. 7A shows that the percentage of molecules that experience the tethering force influences final alignment of the system. When two-thirds of the molecules in a 25-monomer system were exposed to the force, the molecules were unable to align, highlighting the importance of tethering.

FIG. 7B shows that the percentage of molecules that experience the tethering force influences final alignment of the system. When two-thirds of the molecules in a 625-monomer system were exposed to the force, the monomers achieved a certain degree of alignment.

FIG. 7C shows that the percentage of molecules that experience the tethering force influences final alignment of the system. When only one-third of the molecules in a 625-monomer system were exposed to the force, alignment was worse than in FIG. 7B.

FIG. 8A shows an SEM image showing fibrils formed when a neutralized COL solution was left to partially polymerize while exposed to no shear in the protein reservoir for zero minutes. Scale bar=2 μm.

FIG. 8B shows an SEM image showing fibrils formed when a neutralized COL solution was left to partially polymerize while exposed to no shear in the protein reservoir for one minute. Scale bar=2 μm.

FIG. 8C shows an SEM image showing fibrils formed when a neutralized COL solution was left to partially polymerize while exposed to no shear in the protein reservoir for five minutes. Scale bar=2 μm.

FIG. 9A shows a measured histogram illustrating the distribution of fibrils formed under the conditions used to obtain the SEM image of FIG. 8A. The red box illustrates the ±20° range that was utilized to quantify fibril alignment in FIG. 10A.

FIG. 9B shows a measured histogram illustrating the distribution of fibrils formed under the conditions used to obtain the SEM image of FIG. 8B. The red box illustrates the ±20° range that was utilized to quantify fibril alignment in FIG. 10A.

FIG. 9C shows a measured histogram illustrating the distribution of fibrils formed under the conditions used to obtain the SEM image of FIG. 8C. The red box illustrates the ±20° range that was utilized to quantify fibril alignment in FIG. 10A.

FIG. 10A shows fibril alignment analysis of fibrils formed under the conditions used to obtain the SEM images of FIGS. 8A-8C, where n=15 and five measurements were on taken on three independent samples, the error is reported as ±SD, and an asterisk indicates that the results were significantly different from each other (p<0.05).

FIG. 10B shows average fibril diameter in the scaffolds formed under the conditions used to obtain the SEM images of FIGS. 8A-8C, where n=300 fibrils over three independent samples, and the error is reported as ±SD.

FIG. 11 is a graph showing that shear rate was increased to maintain a constant shear stress in the system when scaffolds were produced at three concentrations, 0.5 mg/mL, 1.5 mg/mL, and 2.5 mg/mL of COL, which generated three distinct viscosity profiles.

FIG. 12A shows fibril alignment analysis of scaffolds produced under the conditions used to obtain the SEM images of FIGS. 13A-13C, where n=15 and five measurements were taken on three independent samples, and error is ±SD.

FIG. 12B shows fiber diameter of fibrils in the scaffolds produced under the conditions used to obtain the SEM images of FIGS. 13A-13C, where n=300 fibrils over three independent samples and the error is ±SD. The results depicted in FIG. 12B show that neither the varying concentrations of COL, nor the increased shear rate significantly influenced fibril diameter.

FIG. 12C shows average degrees from central angle of fibrils in the scaffolds produced under the conditions used to obtain the SEM images of FIGS. 13A-13C.

FIG. 13A is an SEM image of fibrils produced from 0.5 mg/mL COL. Scale bar=μm.

FIG. 13B is an SEM image of fibrils produced from 1.5 mg/mL COL. Scale bar=5 μm.

FIG. 13C is an SEM image of fibrils produced from 2.5 mg/mL COL. Scale bar=μm.

FIG. 14A is a representative cross-sectional SEM image of a three-layer COL scaffold. Scale bar=1 μm.

FIG. 14B is a representative cross-sectional SEM image of a five-layer COL scaffold. Scale bar=1 μm.

FIG. 14C is a representative cross-sectional SEM image of a seven-layer COL scaffold. Scale bar=1 μm.

FIG. 15 is a graph showing a comparison of the thickness of three-, five-, and seven-layer scaffolds, such as those depicted in FIGS. 14A-14C, illustrating a significant increase in the thickness of the scaffold, indicating that the scaffold thickness can be increased without negatively influencing fibril organization. The final result is n=75, where three independent scaffolds were prepared for each condition, and five images were taken of each scaffold. Five measurements were then taken on each image and the error reported as ±SD. Results with an asterisk between them indicates a significant difference between values (p<0.05).

FIG. 16 shows fibril alignment analysis results, which indicate that the anisotropic nature of the fibrils was preserved in a larger scaffold produced by a larger device designed and used to produce a scaffold with a 50% increase in the length and width of the scaffold (4.5×1.5 cm) compared to the original one (3.0×1.0 cm). In this analysis, n=5, where five measurements were taken across a single scaffold and error is ±SD.

FIG. 17A is a graphic showing that a COL molecule (monomer) consists of two al chains and one α2 chain assembled as a triple helix. The native molecule has a reported isoelectric point range between 7.0 and 8.3.

FIG. 17B is a graphic showing COL monomers arrange in a quarter-staggered pattern, creating regions of gaps (yellow) and overlaps (orange) denoted as a D-band with an average length of 67 nm.

FIG. 18 is a graphic showing that the mechanism of fibrillogenesis is triggered by the displacement of water molecules. The resulting COL fibrils range from about 50 to about 200 nm in diameter.

FIG. 19 is a graph showing the optical density of assembling COL monitored at 313 nm every 30 second for one hour for the indicated concentrations of COL. Three biological replicates were done, each with three technical replications, for a total of n=9.

FIG. 20 is a graphic showing the protocol of the steps required to produce aligned scaffolds. In the protocol depicted in FIG. 20, a stainless steel spindle was first coated in a layer of gelatin (red) and then placed in a reservoir filled with neutralized monomeric COL. After 15 minutes of spinning, the COL was fully polymerized as a scaffold (gold) that adhered to the gelatin coating on the spindle. To remove the scaffold, the spindle was lowered into a heated solution (blue) to dissolve the gelatin, thus allowing the COL to slide off the surface. An incision was made down the scaffold to open it up into a sheet.

FIG. 21A is a photograph of a five-layer COL scaffold prepared without the addition of a gelatin layer.

FIG. 21B is a photograph of a five-layer COL scaffold prepared with a 10% (w/v) gelation solution at 45° C., and shows an approximately 10-fold increase in the width of the COL scaffold compared to that depicted in FIG. 21A. Scale bar=1 cm.

FIG. 21C is a graph showing thickness of scaffolds prepared with 5%, 10%, or 15% w/v gelation solution at 40° C., 45° C. and 50° C. The 5% (w/v) gelatin produced a thin coating with holes. The 15% (w/v) gelatin solution generated thicker coatings with a greater variation in thickness. As a result, the 10% gelatin was chosen; the 45° C. was the chosen temperature since it led to the coatings with the smallest standard deviation.

FIG. 22 is a comparison of the porosity of the 3-, 5-, and 7-layered COL scaffolds depicted in FIGS. 14A-14C. Results illustrate that there is overall little porosity in the scaffolds and that there is no significant difference in scaffold porosity as the number of layers on the scaffold increases. For the analysis, three replicates of each condition were prepared, fives images were analyzed for each replicate, and five different regions in each image were measured (n=75 per condition). Data presented as the average±SD, and statistical analysis was done with a one-way ANOVA test where p<0.05.

FIG. 23 shows D-banding analysis of fibrillar COL in a scaffold made in accordance with the instant disclosure, rat tail tendons, and a COL gel that formed under no shear. Results indicated that the D-banding frequency on the fibrils in each type of material was statistically similar to each other, and comparable to the 67 nm frequency value that is often reported for native COL. Three biological replicates were prepared for each condition, five images were analyzed for each replicate, and five measurements were taken on each image (n=75 per condition). Results are presented as average±SD, and statistical analysis was done with a one-way ANOVA test where p<0.05.

FIG. 24A is a graph showing the computational analysis of the effect of a force of kcal mol⁻¹ Å⁻¹ on single COL monomer alignment tested on a single coarse-grained simulated COL monomer to determine the minimum amount of forced needed to orient the molecule.

FIG. 24B is a graph showing the computational analysis of the effect of a force of kcal mol^{-Å}⁻¹ on single COL monomer alignment tested on a single coarse-grained simulated COL monomer to determine the minimum amount of forced needed to orient the molecule.

FIG. 24C is a graph showing the computational analysis of the effect of a force of kcal mol^{-1 Å}⁻¹ on single COL monomer alignment tested on a single coarse-grained simulated COL monomer to determine the minimum amount of forced needed to orient the molecule.

FIG. 24D is a graph showing the computational analysis of the effect of a force of 0.005 kcal mol⁻¹ Å⁻¹ on single COL monomer alignment tested on a single coarse-grained simulated COL monomer to determine the minimum amount of forced needed to orient the molecule.

FIG. 25A is an SEM image showing the effect of spinning COL after 10 minutes of polymerization under no shear. Prior to spinning, the spindle remained stationary for 10 minutes. This delay allowed for COL to polymerize before experiencing any shear force. The representative SEM image illustrates the consequences of allowing the COL to polymerize before being spun. When the start time was delayed for 10 minutes, the COL was unable to adhere to the gelatin on the spindle, which further highlighted the necessity for the COL to fully polymerize while exposed to shear. Scale bar=2 μm.

FIG. 25B shows a histogram analysis illustrating the consequences of allowing COL to polymerize before being spun. Results indicated that only 32.0±2.2% of fibrils fell within ±20° of the orientation tensor, which was significantly different from the scaffolds that were not allowed to polymerize (p<0.05).

FIG. 26A is a representative SEM image of a COL scaffold prepared at a shear rate of 50 Scale bar=5 μm.

FIG. 26B is a representative SEM image of a COL scaffold prepared at a shear rate of 75 Scale bar=5 μm.

FIG. 26C is a representative SEM image of a COL scaffold prepared at a shear rate of 100 s⁻¹. Scale bar=5 μm.

FIG. 26D is a representative SEM image of a COL scaffold prepared at a shear rate of 1000 s⁻¹. Scale bar=5 μm.

FIG. 27A shows fibril alignment analysis of scaffolds produced at the indicated shear rates. Results indicated that there was no significant difference in alignment of the scaffolds (p>0.05). When the shear rate of the system surpassed 1000 s⁻¹, the gelatin would rip off of the spindle and no network would form.

FIG. 27B shows fibril diameter analysis of scaffolds produced at the indicated shear rates. Results indicated that there was no significant difference in diameter of the scaffolds (p>0.05).

FIG. 27C is a graph showing the average degrees from the central angle at the indicated shear rates.

FIG. 28A is a photograph of a scaffold (2.5 mg mL⁻¹ COL, 5 layers, 75 s⁻¹ shear rate) broken into nine separate regions. Scale bar=1 cm.

FIG. 28B is a graph showing consistency of fibril alignment throughout the COL scaffold, as a function of region depicted in FIG. 28A. Five images were taken in the each region. The alignment for one region was then measured, averaged, and compared to the alignment in the eight other regions. The top left region was slightly more aligned than other regions on the scaffold, except for the top middle region (p<0.05). However, the alignment in the other regions was not significantly different from each other, highlighting the scaffold uniformity.

FIG. 29 shows fibril alignment analysis of scaffolds comprising three-, five-, and seven-layers of COL. There was a significant increase in fibril orientation variation in the five-layer scaffold compared to the three-layer one. However, there was no difference in alignment between the five- and seven-layer scaffolds, which is why five-layer scaffolds were used for the majority of the experiments. These results indicate that the thickness of the scaffold can be increased without compromising the fibril alignment. Statistically significant results are denoted by an asterisk between conditions where p<0.05.

FIG. 30A is a fluorescent image of the COL scaffold after a 1:5 molar ratio of fibronectin (FN):COL was spun with the final COL layer. Results suggest FN incorporation into the scaffold. Scale bar=200 μm.

FIG. 30B is a fluorescent image of the scaffold of FIG. 30A after being incubated in 2% (v/v) sodium deoxycholate to remove globular fibronectin (FN), revealing that fibrillar FN was present in the scaffold. Scale bar=200 μm.

FIG. 30C is a photograph of the standard scaffold made with the original device next to a scaffold prepared with the device with the larger spindle and protein reservoir.

FIG. 30D is a graph showing indicating that the anisotropic nature of the fibrils was preserved in the larger scaffold and not significantly different from the fibril alignment in the standard scaffolds.

FIG. 31A is a photograph of a scaffold under no strain.

FIG. 31B is a photograph of a scaffold after breaking.

FIG. 32A is a graph showing the maximum force (N) the scaffold and tendons endured before breaking.

FIG. 32B is a graph showing the strain percentage the scaffold and tendons endured before breaking.

FIG. 33 is a graph showing a comparison of the fibril orientation of a three-, five-, and seven-layer scaffold.

FIG. 34A is a graph showing the results of a DC assay of the storage buffer the scaffold was in at 37° C.

FIG. 34B is a graph showing FTIR spectrums of the stored scaffold over time.

FIG. 34C is photograph depicting an SDS-PAGE gel of the buffer storage solution over the 30-day period.

### DETAILED DESCRIPTION

This disclosure is based, at least in part, on the development of a device designed to produce organized, uniaxial fibrillar networks. This device is composed of two concentric cylinders, a rotating inner cylinder powered by a motor, and a stationary outer cylinder. The device generates a shear stress on the solution added to the reservoir. In proof of concept experiments, type I collagen, a self-assembling protein, was used. When the liquid form of the protein was added to the reservoir, the shear stress from the device oriented the developing fibrils into a solid, fibrous material with fibril alignment in the direction of the flow. These fibrils adhered to a sacrificial coating on the spindle, producing a 3×1 cm network of aligned fibrils. To remove the scaffold, the spindle was submerged in warm buffer solution to dissolve the sacrificial layer and remove the scaffold.

The ability to rapidly and reproducibly fabricate anisotropic collagenous networks has remained a challenge for many years. One common approach involved extruding concentrated collagen as fibers with the aid of electrical fields, centrifugal forces, differential solvent baths, shear flow, or 3D-printing. However, some of these methods lack the scalability needed to prepare usable tissue replicates. Other methods can produce fibrils that lack the native ultrastructure of the fibril. To address scalability, a technique that originally was used to make sausage casings known as counter-rotating extrusion (CRE) has been used to make aligned collagen networks at the centimeter scale (Hoogenkamp, H. R. et al., Acta Biomaterialia, 12, 2015, 113-121) (Yang, S. et al., Biomaterials, 207, 2019, 61-75). However, CRE requires significant preparation of the raw material, making it a more inefficient technique.

In an example embodiment, an efficient, compact system that enables robotic control into precision tissue engineering has been designed. In addition, the size of the components of the device can be scaled up to increase the dimensions of the final network, which can be beneficial in tissue engineering applications. Furthermore, the setup allows for integration of other proteins into the network, creating multi-component systems and improving network biocompatibility.

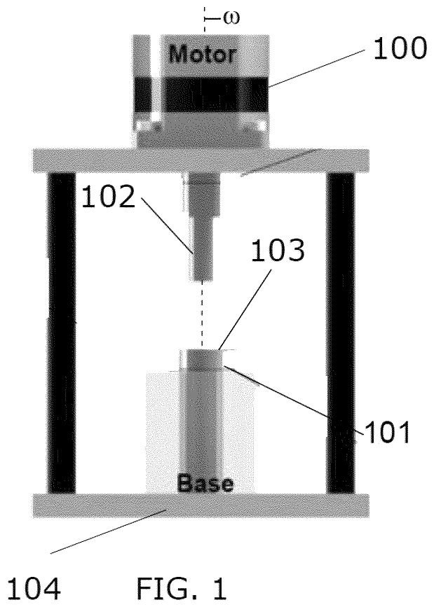

Thus, a small, tabletop system that can rapidly produce highly aligned networks of fibrils at the centimeter scale has been established. This device consists of two concentric cylinders. The inner cylinder, the spindle, is connected to a motor with a speed range of about 50-4000 rpm. The outer cylinder, the protein reservoir, is connected to a base that allows the reservoir to be raised and lowered so the spindle can be submerged in the reservoir. The shear force caused by the spindle rotating in the reservoir orients the fibrils in the direction of the flow during the assembly process. An illustration of the platform can be seen in FIG. 1.

The Spindle: The spindle may be made of stainless steel and secured to the motor with a set screw. This makes it easy to remove the spindle from the system so the formed network can be easily removed. The spindle can also be coated in other proteins or polymers to aid in adhesion to the spindle to ensure that the maximum dimensions of the network are achieved. The dimensions of the spindle can be scaled-up to alter the size of the produced scaffolds.

The Reservoir: The reservoir may also be composed of stainless steel, and holds the protein solution. This component fits into the base that keeps the reservoir in place. The reservoir can be moved up and down in the base in order for the easy addition of material to the reservoir and placement of the spindle. The dimensions of the protein reservoir can also be easily adjusted to change the size of the scaffold.

The Base: The base may be a heavy piece of steel that holds the reservoir in place. The base prevents the reservoir from moving while the spindle is rotating inside of it and allows for easy movement of the reservoir. Example features of example embodiments described herein include:
1. Rapid fibril alignment: The platform can guide fibrils into a uniaxial arrangement while maintaining the natural ultrastructure of the starting material;
2. Network scalability: The dimensions of the individual components, the spindle and the reservoir, can be altered in order produce customized aligned networks;
3. Production of hybrid materials: The platform can be used to incorporate multiple proteins or polymers into a network simultaneously; and/or
4. Incorporation of a sacrificial layer that triggers binding and growth of the fibril network: The use of a sacrificial layer (e.g., made of gelatin) enables these systems to reproducibly form fiber scaffolds.

Examples advantages of example embodiments described herein include:
1. Cost and material accessibility: The majority of the platform is composed of either stainless steel or aluminum, making it relatively inexpensive. The motor is commercially available;
2. Efficiency: The system can align fibrils with little sample preparation, and scaffolds can be prepared in under 2 hours;
3. Design simplicity: Unlike other techniques, the platform does not require the use of harsh solvents, pumps, electric or magnetic fields, or printers to align fibrils. The system is powered solely by a single brushless motor that is commercially available.
4. The components of our system are made of stainless steel and can be easily fabricated in a machine shop. Furthermore, the dimensions of these components can be altered to make a customized end product;
5. The platform produces scaffolds at the centimeter-scale while maintaining the native ultrastructure of collagen fibrils; and/or
6. The platform enables incorporation of multiple materials into the networks to create hybrid scaffolds.

Example uses of example embodiments described herein include:
1. Tissue engineering: Fibril alignment is a critical component in native tissue and aids in the function of the tissue. The system produces networks of aligned fibrils that can be utilized as tissue grafts;
2. In vitro platforms: Networks that replicate the native extracellular matrix can play a beneficial role in cell studies;
3. Regenerative medicine: The networks have the potential to replace protein-dense tissue in the body, such as tendons and ligaments;
4. Textile design: The system is not only amenable to proteins. It is believed that it can be used to polymerize and grow highly aligned polymers with potential applications in textile design; and/or
5. Biomimetic in vitro tissue platforms for cell studies or tissue therapies.

A description of further example embodiments follows.

### Compositions

Provided herein is a fibrillar polymeric scaffold. Also provided herein is a composition comprising a fibrillar polymeric scaffold. The scaffolds and compositions described herein can be prepared in whole or in part by the methods described herein. Thus, also provided herein is a fibrillar polymer scaffold and/or composition comprising a fibrillar polymeric scaffold produced according to any of the methods described herein.

As used herein, “fibrillar polymeric scaffold” refers to a three-dimensional structure comprising polymeric fibrils.

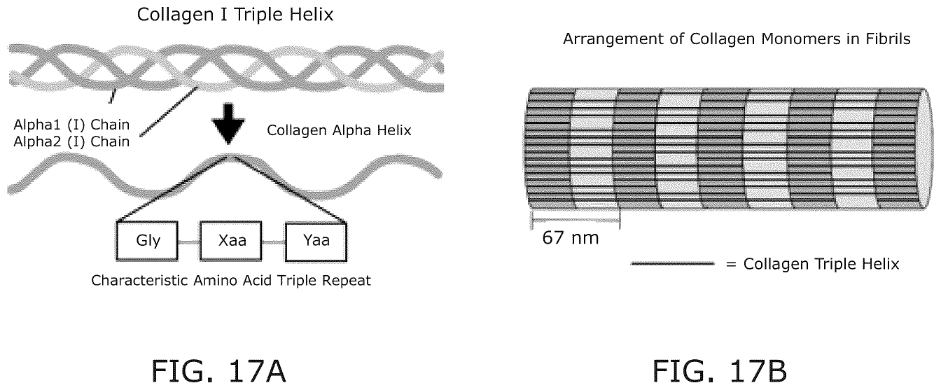

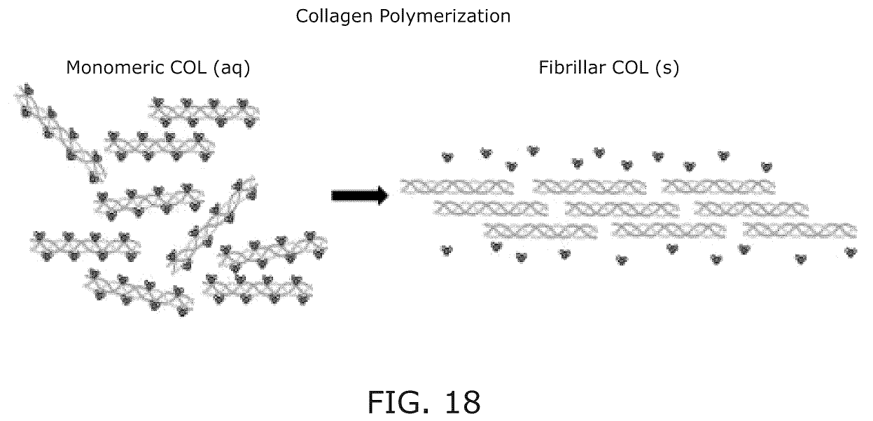

In some embodiments, the fibrils intertwine within the scaffold, e.g., as depicted in FIG. 17A. The fibrils include, in some embodiments, D-banding, such as D-banding present in native COL fibrils, which typically have an average length of 65.5+/−3.6 nm.

Typically, a fibrillar polymeric scaffold described herein comprises fibrils (e.g., a plurality of fibrils) derived from a polymerizable monomer. In an embodiment, the polymerizable monomer is a fiber forming monomer. In some embodiments, the fiber forming monomer is cotton, wool, polyester, polyamide, polyacrylonitrile, rayon, collagen, actin, fibrin, or lignin. In preferred embodiments, the fiber forming monomer is collagen. In a particular embodiment, the fiber forming monomer is type I collagen.

As used herein, “fiber forming monomer” refers to a polymerizable monomer, which, upon polymerization under appropriate conditions (such as those disclosed herein), form fibers (e.g., fibrils). Examples of fiber forming monomers include, but are not limited to, regenerated cellulose, cellulose triacetate, polycaprolactam (textile fibre), polyhexamethylene adipamide (textile fibre), polycaprolactam (industrial fibre), polyhexamethylene adipamide (industrial fibre), poly-p-phenylene terephthalamide, polyethylene terephthalate, and/or acrylic (>85% acrylonitrile).

In an embodiment, the fibrillar polymeric scaffold is derived from one or more additional polymerizable monomers, e.g., one or more additional fiber forming monomers. In some embodiments, the one or more additional fiber forming monomers is a fibral protein. In some embodiments, the fibral protein is zein, decorin, hyaluronic acid, cellulose, lamin, fibrinogen, fibrin, or fibronectin. In a particular embodiment, the fibral protein is fibronectin.

Advantageously, the methods described herein produce fibrillar polymeric scaffold that are anisotropic. Accordingly, in some embodiments, the fibrillar polymeric scaffold is anisotropic. Anisotropy can be assessed, e.g., using fibril alignment analysis, such as that described in the Exemplification. For example, the alignment of the fibrils can be measured as a “P₂ value.” P₂ is a qualitative measure that fluctuates between perfectly aligned (value of 1) and entirely disordered (value of 0); a greater number generally indicates greater alignment. P₂ values are conveniently converted to percentages by multiplying the P₂ value by 100.

In an embodiment, about 50% or greater of the fibrils are within ±20° of the orientation of all fibrils in the scaffold. In an embodiment, about 60% or greater of the fibrils are within ±20° of the orientation of all fibrils in the scaffold. In an embodiment, about 70% or greater of the fibrils are within ±20° of the orientation of all fibrils in the scaffold. In an embodiment, about 80% or greater of the fibrils are within ±20° of the orientation of all fibrils in the scaffold. In an embodiment, about 90% or greater of the fibrils are within ±20° of the orientation of all fibrils in the scaffold.

In an embodiment, the fibrils exhibit D-bands. As used herein, “D-bands” or “D-periods” refers to the tropocollagen subunits that spontaneously self-assemble, with regularly staggered ends, into even larger arrays in the extracellular spaces of tissues. In the native fibrillar collagens, molecules are staggered to adjacent molecules by about 67 nm. In each D-period repeat of the microfibril, there is a part containing five molecules in cross-section, called the “overlap”, and a part containing only four molecules, called the “gap”. These overlap and gap regions are retained as microfibrils assemble into fibrils, and are viewable using electron microscopy.

In an embodiment, the fibrils have a substantially uniform diameter. In an embodiment, the fibrils have a diameter of from about 50 nm to about 200 nm, for example, from about 125 nm to about 160 nm. In some embodiments, the fibrils have a diameter of about 125, about 130, about 135, about 140, about 145, about 150, about 155, or about 160 nm.

The fibrillar polymeric scaffolds disclosed herein can include one or more layers, as explained in more detail herein. Thus, in some embodiments, the scaffold is a one-layer scaffold. In some embodiments, the scaffold is a multi-layer scaffold (e.g., from 2 to 100 layers, from 2 to 50 layers, from 2 to 25 layers, from 2 to 15 layers, from 2 to 10 layers, or 2, 3, 4, 5, 6, 7, 8, 9, 10, 11, 12, 13, 14, or 15 layers). When the scaffold is a multi-layer scaffold, the layers can be the same or different from one another, e.g., in composition, fibril alignment, fibril diameter, porosity and/or thickness. Thus, for example, in an embodiment, about 50% or greater of the fibrils of a particular layer are within ±20° of the orientation of all fibrils in the layer, e.g., about 60% or greater, about 70% or greater, about 80% or greater, or about 90% or greater of the fibrils of a particular layer are within ±20° of the orientation of all fibrils in the layer. For example, in an embodiment, the fibrils in a layer have a substantially uniform diameter, e.g., a diameter of from about 50 nm to about 200 nm, from about 125 nm to about 160 nm, about 125, about 130, about 135, about 140, about 145, about 150, about 155, or about 160 nm.

In preferred embodiments, each layer is the same or substantially the same as the other layers in the scaffold. In an embodiment, the orientation of the fibrils in each layer of a scaffold is uniform or substantially uniform. In an embodiment, the diameter of the fibrils in each layer of a scaffold is uniform or substantially uniform.

In an embodiment, the orientation of the fibrils in the scaffold is uniform or substantially uniform throughout the fibrillar polymeric scaffold as a function of thickness (e.g., across the layers of a multi-layer scaffold). In an embodiment, the diameter of the fibrils in the scaffold is uniform or substantially uniform throughout the fibrillar polymeric scaffold as a function of thickness (e.g., across the layers of a multi-layer scaffold).

In an embodiment, the fibrillar polymeric scaffold has a thickness of at least about 300 nm. In some embodiments, the fibrillar polymeric scaffold has a thickness of at least about 300, about 400, about 500, about 600, about 700, about 800, or about 900 nm. In some embodiments, the fibrillar polymeric scaffold has a thickness of from about 300 nm to about 900 nm.

The intertwining, fibril orientation, fibril diameter, fibril alignment, D-banding and/or similar structural properties of a fibrillar polymeric scaffold may also be referred to herein as the “microstructure” of the fibrillar polymeric scaffold. The COL-derived fibrillar polymeric scaffolds disclosed herein, for example, mimic or substantially mimic the microstructure of native rat tail tendon.

In an embodiment, the fibrillar polymeric scaffold is non-cellular, meaning it lacks cells.

In another aspect, provided herein is a composition comprising a fibrillar polymeric scaffold, including any of the fibrillar polymeric scaffolds described herein, such as a fibrillar polymer scaffold comprising fibrils derived from one or more polymerizable monomers, and a sacrificial polymerization scaffold, wherein the sacrificial polymerizable scaffold is attached to (e.g., removably attached to, as when the sacrificial polymerizable scaffold can be selectively dissolved in the presence of the fibrillar polymeric scaffold; adhered to) the fibrillar polymeric scaffold. In an embodiment, the sacrificial polymerization scaffold comprises gelatin or a poloxamer (e.g., a layer of gelatin or poloxamer; a coating of gelatin or poloxamer). In a particular embodiment, the sacrificial polymerization scaffold comprises gelatin (e.g., a layer of gelatin; a coating of gelatin).

### Apparatus

Also provided herein is an apparatus for preparing any of the compositions described herein, e.g., using any of the methods described herein. Accordingly, provided herein is an apparatus comprising two concentric cylinders, an outer cylinder (e.g., a stationary outer cylinder) that forms a reservoir having a first radius, and an inner cylinder (e.g., an inner rotating cylinder) configured to be rotated by a motor and having a second radius smaller than the first radius, wherein the outer cylinder and inner cylinder are independently rotatable with respect to one another (e.g., by a motor).

Also provided herein is an apparatus comprising two concentric cylinders, a rotating inner cylinder configured to be rotated by a motor, and a stationary outer cylinder, wherein the outer cylinder forms a reservoir. It will be understood that when a cylinder, such as an inner rotating cylinder, is at rest, it will not be actively rotating, but still rotatable.

By virtue of, inter alfa, its two concentric cylinders independently rotatable with respect to one another (e.g., a stationary outer cylinder and an inner rotating cylinder; a rotating outer cylinder and an inner stationary cylinder; a rotating outer cylinder and an inner rotating cylinder), the apparatus may, in use, apply shear flow to a liquid in the reservoir. Accordingly, in an embodiment, the device is configured to apply shear flow to a liquid in the reservoir.

In an embodiment, the second radius is uniform or substantially uniform along a length of the inner cylinder (e.g., the entire length of the inner cylinder; at least the length of the inner cylinder submerged in liquid contained within the reservoir, e.g., when the apparatus is in use). In another embodiment, the inner cylinder is not tapered, at least along the length of the inner cylinder submerged in liquid contained within the reservoir, e.g., when the apparatus is in use.

In an embodiment, the apparatus further comprises a motor configured to rotate one or both of the concentric cylinders. In a preferred embodiment, the apparatus further comprises a motor configured to rotate the inner cylinder. The motor may be a brushless motor, a brushed motor, a direct drive motor, a linear motor, a servo motor, or a stepper motor, and the revolutions per minute (RPM) of the motor can be fixed or variable. Such motors are commercially available.

In an embodiment, the apparatus further comprises a base attached to the outer cylinder.

To facilitate removal of scaffold formed in the apparatus, at least one of the outer and inner cylinders is perpendicularly movable with respect to the other. For example, in some embodiments, the inner cylinder (e.g., the inner rotating cylinder) is perpendicularly movable with respect to the outer cylinder (e.g., the stationary outer cylinder).

Also to facilitate removal of scaffold formed in the apparatus, at least one of the outer and inner cylinders is removable from the apparatus. Preferably, the removable cylinder corresponds to the cylinder on which the scaffold forms when the apparatus is used to make a scaffold in accordance with this disclosure. In some embodiments, the inner cylinder is removable from the apparatus. In an embodiment, the inner cylinder is attachable to the apparatus, e.g., the motor of the apparatus, by a single screw or other removable affixment implement.

In an embodiment, the inner cylinder and/or the outer cylinder are stainless steel or aluminum. In an embodiment, the inner cylinder and/or the outer cylinder are any corrosive resistant material (e.g., stainless steel, aluminum, titanium, plastic, ceramic, etc.). The examples of building materials for the apparatus are not limited to the materials listed herein. The skilled artisan would be able to ascertain what materials are suitable and/or possible for apparatus construction based on the chemical composition and the mechanical stress of the apparatus.

The ratio of the diameter of the outer cylinder to the diameter of the inner cylinder can be fixed, so as to facilitate creation of a shear flow in a liquid contained in the reservoir when the inner and/or outer cylinders are rotated with respect to one another. In some embodiments, the ratio of the diameter of the outer cylinder to the inner cylinder is about 3 to 1, for example, about 2 to 1, about 1.5 to 1, or about 1.2 to 1. In a preferred embodiment, the ratio of the diameter of the outer cylinder to the inner cylinder is about 1.2 to 1.

FIG. 1 depicts an apparatus in accordance with certain embodiments described herein. The apparatus comprises two concentric cylinders, rotating inner cylinder or spindle 102 configured to be powered by motor 100, and stationary outer cylinder 101 which forms protein reservoir 103. In FIG. 1, motor 100 is configured to power spindle 102 to rotate about axis co. Spindle 102 fits within outer cylinder 103 when spindle 102 is lowered along axis ω into reservoir 103 by virtue of having a smaller radius than protein reservoir 103. In FIG. 1, the stationary outer cylinder 101 is attached to stationary base 104, which provides the apparatus with stability and is convenient for use on a benchtop, for example.

### Methods of Preparation

It has been found that fibrillar polymeric materials (e.g., anisotropic fibrillar polymeric materials, including the fibrillar polymeric materials described herein) can be produced rapidly and reproducibly under mild conditions, e.g., using layer-by-layer assembly as described herein.

Accordingly, provided herein is a method for preparing a fibrillar polymeric scaffold, such as any of the fibrillar polymeric scaffolds described herein, comprising: (a) contacting an adherent solid surface with a mixture comprising a polymerizable monomer and an aqueous solvent under conditions suitable to induce adhesion of polymerizable monomer to the adherent solid surface; and (b) applying shear flow to the mixture under conditions suitable to polymerize the polymerizable monomer into fibrils, thereby preparing the fibrillar polymeric scaffold.

As used herein, “adherent solid surface” refers to a solid surface that induces adhesion of a polymerizable monomer, or a portion thereof, particularly a terminus thereof, to the surface, such that in the presence of a facilitating force, the monomer uncoils and polymerizes. Adhesion to a solid surface, such as a sacrificial layer, may be based on ionic bonding, hydrogen bonding, or covalent bonding. In some embodiments, the adherent solid surface is a porous metal surface. In some embodiments, the adherent solid surface is removable from an apparatus to which is it attached. In some embodiments, the solid surface is a sacrificial or non-sacrificial organic or inorganic layer.

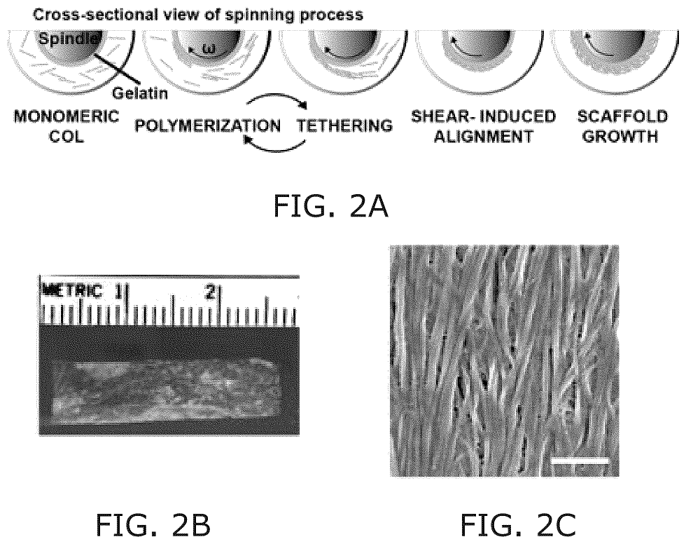

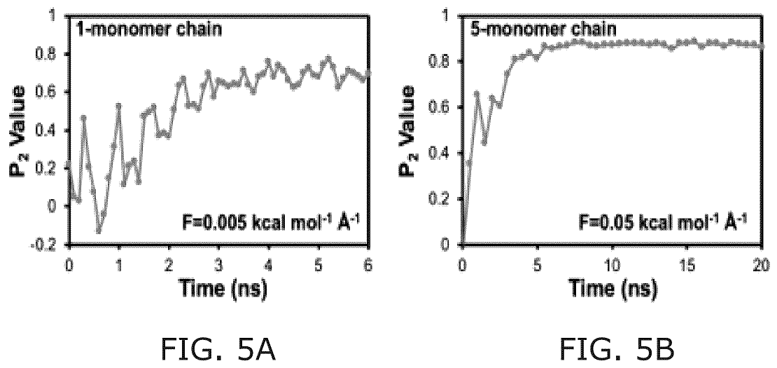

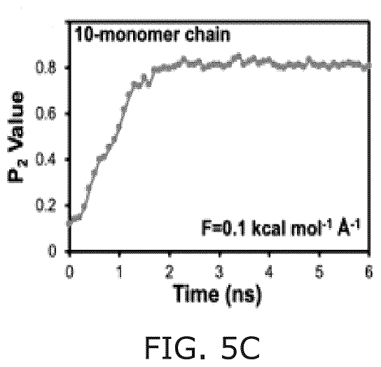

The process by which an adherent solid surface induces adhesion of or tethers a COL monomer described herein is depicted graphically in FIG. 2A. Adhesion or tethering can be initiated by applying a point force at one end of a tropocollagen at the beginning of a polymerization process. Upon application of facilitating force, such as shear flow (e.g., Couette flow), to the solution containing tropocollagen, the tropocollagen is dragged with the flow. Although not wishing to be bound by any particular theory, tethering is thought to cause an initial deformation of the COL molecules experiencing the tethering force, which was defined as the uncoiling and alignment of the molecules on the spindle. It was experimentally observed that a single COL molecule gradually aligned within about 3 ns when the magnitude of the tethering force was above 0.005 kcal/mol/Å (FIG. 5A). When the tethering force was below this threshold, the simulated COL could not overcome the pairwise, long-range cohesive/attractive forces between two interacting molecules. Above this threshold value, the tethering force could surmount these opposing forces and initiate the uncoiling of the COL molecule, which eventually facilitated overall alignment of the structure.

Fibrils are typically characterized by rod-like structures with high length-to-diameter fatios, with diameters being on the nanometer scale. The fibrils formed herein typically have a uniform or substantially uniform cross-section along their lengths.

As used herein, “shear flow” refers to a type of fluid flow in which adjacent layers of fluid move parallel to each other with different speeds. Viscous fluids resist this shearing motion. For a Newtonian fluid, the stress exerted by the fluid in resistance to the shear is proportional to the strain rate or shear rate.

In some embodiments, the shear flow is laminar. As used herein, “laminar” refers to a type of fluid (gas or liquid) flow in which the fluid travels smoothly or in regular paths.

In some embodiments, the shear flow is Couette flow. As used herein, “Couette flow” refers to the flow of a viscous fluid in the space between two surfaces, one of which is moving tangentially relative to the other. Such a flow is created in the apparatuses described herein when the outer cylinder and the inner cylinder move tangentially relative to one another.

In some embodiments, the shear flow has a shear rate of from about 50 s⁻¹ to about 1000 s⁻¹. In some embodiments, the shear flow has a shear rate of from about 50 s⁻¹ to about 900 s⁻¹, from about 50 s⁻¹ to about 800 s⁻¹, from about 50 s⁻¹ to about 700 s⁻¹, from about 50 s⁻¹ to about 600 s⁻¹, from about 50 s⁻¹ to about 500 s⁻¹, from about 50 s⁻¹ to about 400 s⁻¹, from about 50 s⁻¹ to about 300 s⁻¹, from about 50 s⁻¹ to about 200 s⁻¹, or from about 50 s⁻¹ to about 100 s -1 . In a particular embodiment, the shear rate is from about 50 s⁻¹ to about 100 s⁻¹.

In an embodiment, the shear flow exerts a shear stress on the mixture of from about 0.5 N/m² to about 15 N/m². In some embodiments, the shear flow exerts a shear stress on the mixture of from about 0.7 N/m² to about 14 N/m², about 1.0 N/m² to about 14 N/m², about 2.0 N/m² to about 13 N/m², about 3.0 N/m² to about 12 N/m², about 4.0 N/m² to about 11 N/m², about 5.0 N/m² to about 10 N/m², about 6.0 N/m² to about 9.0 N/m², or about 7.0 N/m² to about 8.0 N/m². In some embodiments, the shear flow exerts a shear stress on the mixture of about 0.5, about 0.6, about 0.7, about 0.8, about 0.9, about 1.0, about 1.1, about 1.2, about 1.3, about 1.4, about 1.5, about 2.0, about 3.0, about 4.0, about 5.0, about 6.0, about 7.0, about 8.0, about 9.0, about 10, about 11, about 12, about 13 or about 14 N/m². In a particular embodiment, the shear stress is about 1.1 N/m².

In an embodiment, the mixture has a viscosity of from about 1.0 cP to about 25 cP. In an embodiment, the mixture has a viscosity of from about 1.0 cP to about 25 cP, about 2.0 cP to about 19 cP, about 3.0 cP to about 18 cP, about 4.0 cP to about 17 cP, about 5.0 cP to about 16 cP, about 6.0 cP to about 15 cP, about 7.0 cP to about 14 cP, about 8.0 cP to about 13 cP, about 9.0 cP to about 12 cP, about 10 cP to about 11 cP. In some embodiments, the mixture has a viscosity of about 1.0, about 2.0, about 3.0, about 4.0, about 5.0, about 6.0, about 7.0, about 8.0, about 9.0, about 10, about 11, about 12, about 13, about 14, about 15, about 16, about 17, about 18, about 19, or about 20 cP.

Also provided herein is a method for preparing a fibrillar polymeric scaffold, such as any of the fibrillar polymeric scaffolds described herein, in an apparatus, e.g., any of the apparatuses described herein, the method comprising: (a) providing a mixture comprising a polymerizable monomer and an aqueous solvent in a reservoir under conditions suitable to induce adhesion of polymerizable monomer to an adherent solid surface in contact with the reservoir; and (b) applying shear flow to the mixture under conditions suitable to polymerize the polymerizable monomer into fibrils, thereby preparing the fibrillar polymeric scaffold. In some embodiments, the apparatus comprises two concentric cylinders, an outer cylinder that forms the reservoir having a first radius, and an inner cylinder having a second radius smaller than the first radius, wherein the outer cylinder and inner cylinder are independently rotatable with respect to one another, and the outer cylinder or inner cylinder comprises the adherent solid surface.

In an embodiment, the apparatus comprises two concentric cylinders, a stationary outer cylinder that forms a reservoir having a first radius and a rotating inner cylinder having a second radius smaller than the first radius, wherein the rotating inner cylinder comprises an adherent outer solid surface.

In an embodiment, (a) and (b) are simultaneous or nearly simultaneous. It has been found that when significant polymerization is allowed to occur in the absence of shear flow, the resulting fibrillar polymeric scaffolds suffer from lower levels of fibril alignment and anisotropy. Stated otherwise, it is often desirable to begin applying shear flow to a mixture of polymerizable monomer that does not contain an appreciable or significant amount of polymerization product. Thus, in an embodiment, the mixture does not contain polymerized monomer, or contains less than about 25%, e.g., less than about 20%, less than about 15%, less than about 10%, less than about 5%, less than about 2%, or less than about 1%, polymerized monomer, based on the initial amount of polymerizable monomer, e.g., when shear flow is initially applied to the mixture.

In an embodiment, applying shear flow to the mixture comprises rotating the inner cylinders, the outer cylinder, or both with respect to one another to generate the shear flow. In an embodiment, applying shear flow to the mixture comprises rotating the inner cylinder with respect to the outer cylinder to generate the shear flow. In an embodiment, the inner and outer cylinders are further configured to move perpendicularly to one another.

In an embodiment, the difference between the first radius and the second radius is from about 0.5 mm to about 3 mm. In embodiment, the difference between the first radius and the second radius is about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 mm. In a particular embodiment, the difference between the first radius and the second radius is about 1.0 mm.

As used herein the term “about” is used herein to mean approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower), e.g., 15 percent up or down, 10 percent up or down, 5 percent up or down, 4 percent up or down, 3 percent up or down, 2 percent up or down, or 1 percent up or down.

When an element, such as fibril diameter, is described as “substantially the same” or “substantially uniform” herein, the element is about the same or about uniform, where “about” is as described herein.

In the methods described herein, the scaffolds typically form on the adherent solid surface. In some embodiments, the scaffold is formed on a sacrificial polymerization scaffold. In an embodiment, the adherent solid surface comprises a sacrificial polymerization scaffold. In an embodiment, the sacrificial polymerization scaffold is or comprises gelatin or a poloxamer (e.g., a gelatin coating, a poloxamer coating). In a particular embodiment, the sacrificial polymerization scaffold is or comprises a gelatin coating.

In an embodiment, the scaffold is formed on a fibrillar polymeric scaffold, e.g., as when adding subsequent layers to a pre-existing fibrillar polymeric scaffold. In an embodiment, the adherent solid surface comprises a fibrillar polymeric scaffold (e.g., a pre-formed fibrillar polymeric layer). In a particular embodiment, a first layer of fibrillar polymeric scaffold is formed on a sacrificial or non-sacrificial polymerization scaffold, and subsequent layers are formed on the resulting fibrillar polymeric scaffold. In an embodiment, the scaffolds described herein comprise 1, 2, 3, 4, 5, 6, 7, 8, 9, 10, 11, or 12 layers.

In an embodiment, the method further comprises repeating (a) and (b) n times, wherein n is an integer from 2 to 25. In some embodiments, n is 2, 3, 4, 5, 6, 7, 8, 9, 10, 11, 12, 13, 14, 15, 16, 17, 18, 19, 20, 21, 22, 23, 24, or 25. In particular embodiments, n is 3, 4, 5, 6, 7, 8, 9, or 10. In an embodiment, each iteration creates a new layer of the scaffold. The integer n corresponds to the number of “layers” in the scaffold. In some embodiments, the number of layers (i.e., n) is between 5 and 10. Other suitable numbers of layers are as described elsewhere herein.

In an embodiment, the method further comprises removing the fibrillar polymeric scaffold from the adherent solid surface. In some embodiments, the fibrillar polymeric scaffold can be removed from the adherent solid surface via mechanical means. In some embodiments, the fibrillar polymeric scaffold can be removed from the adherent solid surface via chemicals means, e.g., by dissolving the adherent solid surface in a phosphate buffer solution, as, for example, when the adherent solid surface comprises gelatin.

### EXEMPLIFICATION

### Example 1. The Rapid Production of Highly Anisotropic Fibrillar Collagen Scaffolds

### Materials and Methods

### Analysis of the Sacrificial Gelatin Coating

To determine the optimal conditions required to produce a thin, consistent gelatin coating on the spindle, 5,10, and 15% (w/v) solutions of 300 bloom type A gelatin powder (Electron Microscopy Sciences, 9000-70-8) were prepared in a diluted phosphate buffered saline solution (1×PBS) (Fisher Scientific, BP3991) via stirring and heating at 37° C. The 1×PBS solution was composed of 0.0119 M phosphate buffer, 0.137 M sodium chloride, and M potassium chloride. Once dissolved, the gelatin solution was heated in a water bath to either 40, 45, or 50° C., for a total of nine different conditions to test.

In order to remove the gelatin from the stainless-steel spindle in one piece, the spindle was first coated in a 20% (w/v) solution of Pluronic® F-417 (Sigma Aldrich, P2443). This material was chosen because it is a liquid below room temperature and a solid above room temperature, the opposite of gelatin; the Pluronic® F-417 serves as a sacrificial layer and allows us to remove the gelatin coating in one piece. The Pluronic® F-417 solution was prepared in 1×PBS solution, and was stirred at 4° C. until completely dissolved. The spindle was chilled to 4° C. to prevent the Pluronic® F-417 from spontaneously gelling on the spindle and was dipped into the 4° C. Pluronic® F-417 solution. The spindle was then spun sideways at room temperature at 75 s⁻¹ for 30 minutes so the solution could fully dry. Next, the spindle was dipped into the heated gelatin for about 10 seconds, slowly removed, and then rotated sideways at about 75 s′ for 10 minutes so the protein could gel. The spindle was then placed in 4° C. distilled water to dissolve the Pluronic® F-417, allowing the gelatin coating to slide off the spindle. An incision was made down the gelatin coating to open it up and lay it flat on a glass slide for analysis.

A glass needle was utilized to locate the surface of the gelatin at five different locations, and then the distance between the top of the microscope slide and the surface of the gelatin was determined to be the thickness of the coating. The gelatin was kept hydrated for the entire analysis process and this process was repeated three times for each condition.

The gelatin was transferred to an inverted microscope (Nikon, ECLIPSE, Ti2) with a 0.3 numerical aperture 10× objective (Nikon, MRH00101) and a CMOS camera (Andor, Zyla 4.2). A glass needle was utilized to determine the thickness of the gelatin. The needle was held in a micromanipulator (Eppendorf, Transferman 4r) that could move the needle in the XYZ directions. Deflection of the needle was used to determine the top of the gelatin and the top of the glass microscope slide. The difference between the two was the thickness of the coating. The gelatin was kept hydrated for the entire analysis process and this process was repeated three times for each condition.

### Preparation of the Sacrificial Gelatin Coating for COL Scaffold

A 10% (w/v) solution of gelatin (Electron Microscopy Sciences, 9000-70-8) was prepared in diluted phosphate buffer saline (1×PBS) via stirring and heating at 37° C. Once dissolved, the solution was heated to 45° C. in a water bath. The spindle was slowly dipped into and removed from the heated gelatin. The spindle was then attached to the motor and spun sideways at 75 s⁻¹ for 10 minutes. This process generated a smooth, uniform coating on the spindle with a thickness of 28.3±5.0 μm.

### Preparation of COL Solutions

Acetic acid-extracted bovine type I collagen (Advanced Biomatrix 5026-1KIT) was mixed and neutralized with concentrated phosphate buffer saline (10×PBS) (Fisher Scientific, BP3991) and sodium hydroxide for a final concentration of either 2.5, 1.5, or 0.5 mg/mL. The 10×PBS was composed of 0.119 M phosphate buffer, 1.37 M sodium chloride, and 0.027 M potassium chloride always made up 10% of the solutions final volume to replicate physiological ionic strengths. The buffer, along with 0.1 M sodium hydroxide was used to adjust the pH of the solution to 7.3±0.1, and then distilled water was used to bring the sample to the required volume.

### Preparation of the COL Scaffold

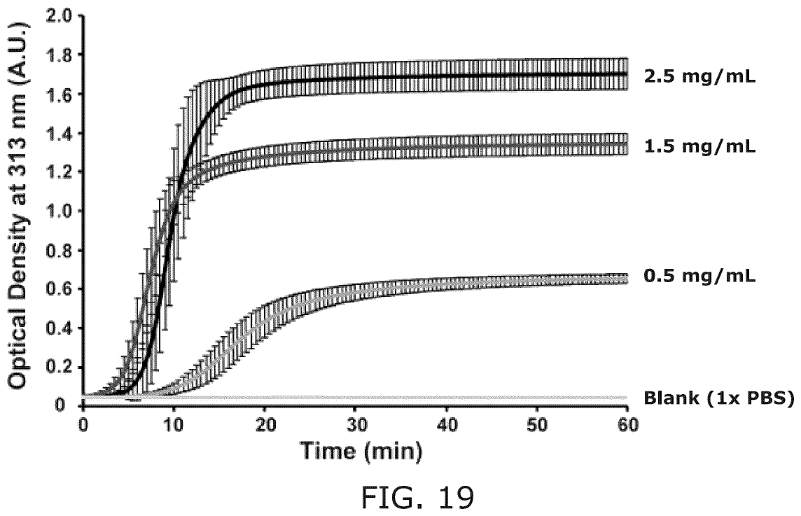

A 0.6 mL solution of the neutralized COL was pipetted into the protein reservoir of the device and the gelatin-coated spindle was positioned into the center of the chamber and submerged in the solution. The motor was turned on to the desired speed, and the solution was left to spin until the COL completely polymerized (FIG. 19). Next, the motor was then turned off, the reservoir was carefully lowered, and the remaining solution was removed. This whole process was repeated multiple times in order to build up the scaffold. Afterwards, a vertical incision was made down the scaffold to make it easier to separate the scaffold from the spindle. The spindle was then submerged in a 37° C. 1×PBS solution until the gelatin coating completely dissolved. The COL network was then able to be carefully slid off of the spindle and opened into a rectangular sheet. If the scaffolds were imaged, they were first fixed for two hours in 2.5% (v/v) glutaraldehyde (in 1×PBS) and then buffer-exchanged by replacing half of the solution volume with 1×PBS solution seven times; this was done to make it easier to prepare the material for imaging. Otherwise, the scaffolds they were stored at 37° C. in 1×PBS with 0.02% (w/v) sodium azide to prevent bacteria growth.

### Preparation of the COL Scaffold

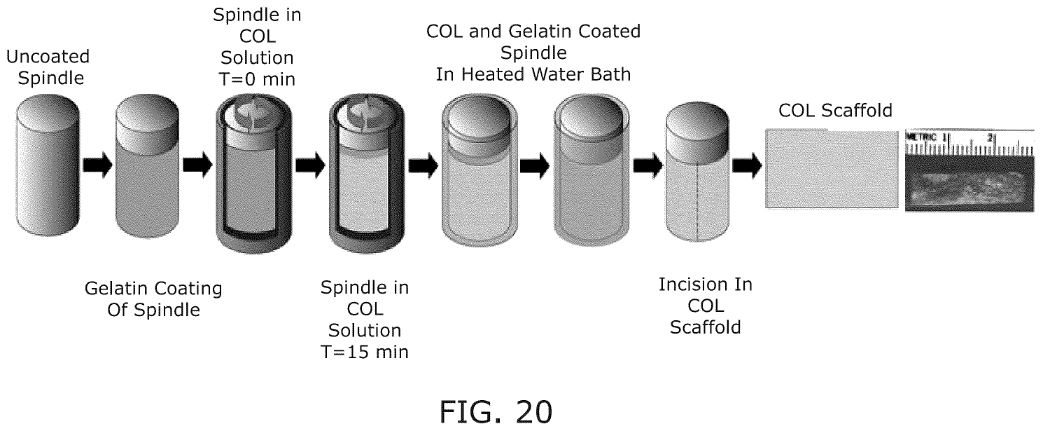

A 0.6 mL solution of the neutralized COL was pipetted into the protein reservoir, and the gelatin-coated spindle was positioned into the center of the chamber and submerged in the solution. The motor was turned on to the desired speed, and the solution was left to spin until the COL completely polymerized at room temperature. The process was consistently performed at 21° C.±2° C. so the gelatin would remain a solid during the process. Afterwards, the reservoir was lowered, and the remaining solution was removed. This whole process was done five times to build up the scaffold. A vertical incision was then made to remove the scaffold from the spindle. The spindle was then submerged in a 37° C. 1×PBS solution to dissolve the sacrificial gelatin layer (FIG. 20). For materials that were prepared under no shear, the collagen was allowed to polymerize in the reservoir while the spindle remained stationary. Multiple spin cycles were not required for this process, as the COL fibrils formed as mass at the bottom of the reservoir that was easy to transfer. Throughout all experiments, a negligible amount of evaporation occurred during a single spin cycle (<0.01%), suggesting the humidity of the surrounding environment should not significantly impact the final product.

### Scanning Electron Microscopy (SEM) Imaging

The scaffold was cut in half and then carefully transferred from the solution onto a stainless-steel SEM stage coated with carbon tape and laid flat. The side of the scaffold that was facing out towards the solution was face-up on the stub. The scaffold was left to air-dry overnight and then sputter coated with 2.5 nm of platinum. The scaffolds were then imaged with a scanning electron microscope (Hitachi, S-4800) to show the fibril organization in the network. Five representative images were then taken at different locations of the scaffold to both obtain an accurate fibril alignment measurement of the scaffold and ensure uniform fibril alignment throughout the scaffold.

### COL Fibrillogenesis Kinetics

Samples of 2.5, 1.5, and 0.5 mg mL⁻¹ COL with a pH value of 7.3±0.1 were prepared with 10×PBS and neutralized with 0.1M sodium hydroxide, and changes in optical density at 313 nm were measured for each condition similar to previous reports. Each condition, along with a blank of 1×PBS, was done three times for a total of n=9.

### Porosity Measurements

To analyze the porosity of the scaffold, a region of an SEM image was focused on and the percentage of pixels below 50 on a grayscale of 0-255 in ImageJ. This process was repeated at five locations on a single image. For each condition, three trials were performed. Five images were taken for each trial and five measurements were taken on each image. The values were averaged together for a final value and the error was reported as ±the standard deviation.

### D-Banding Analysis

To analyze the ultrastructure of the COL fibrils in the scaffolds, rat tail tendons, and COL fibrils in an isotropic array, SEM images were analyzed in ImageJ. A line was drawn across five bands (where one band consists of a gap and an overlap), and the measured value was divided by five to obtain the length of one band. Three trials were performed for each condition and each trial consisted of five images. Five measurements were taken on each image and the values were averaged together for a final value and the error was reported as ±the standard deviation.

### Coarse-Grained Model

This model is derived from a high-fidelity AA model with explicit water solvents, where the hydrodynamic interaction between water and COL will pose significant impact to a series of mechanical behaviors from bending to shearing of the COL. The fluid shear is being handled within this implicit-solvent potential forcefield. Therefore, by fitting the CG potential to match a variety of mechanical behaviors such as various types of mechanical loading including tension, compression, shear, and bending to the high-fidelity water-explicit AA model, the effects of solvation water and the necessary hydrodynamic interactions are taken into account. More importantly, the very flexible and solvent-sensitive COL property, persistence length of tropocollagen, which is usually measured through experiments, can also be accurately captured with this model, validating its utility the COL solution.

### CG Simulation Framework Parameters:

| Parameter | Value |
|---|---|
| Dispersive parameter (σ_{LJ}, Å) | 14.72 |
| Dispersion energy parameter (ε_{LJ}, kcal/mol) | 11.06 |
| Tensile stiffness parameter (k₁, kcal/mol/Å²) | 17.13 |
| Tensile stiffness parameter (k₂, kcal/mol/Å²) | 97.66 |
| Equilibrium angle (β₀, degree) | 170-180 |
| Bending stiffness parameter (k_{angular}, | 14.98 |
| kcal/mol/rad²) | |
| Equilibrium bead distance (r₀, Å) | 14 |
| Critical hyperelastic distance(r₁, Å) | 18.2 |
| Bond breaking distance (r_{break}, Å) | 21 |

### COL Tethering

The tethering is handled by applying a point force at one end of the tropocollagen; wherein a force is applied to one end of the COL in the very beginning, then other portions of the COL will be “dragged” with the facilitating flow from the rotating spindle. Our point force is applied to the bead centroid, which is an infinitesimal entity, where there is no difference between applying tension or shear to this entity. Thus, we assume this point force should mimic the shear applied in experiments in a similar manner. Parameter values mainly include the potential forcefield parameters, tethering threshold forces, and COL molecule numbers. The potential forcefield parameters are set strictly following the previous validated literatures, and the tethering threshold forces are determined through a series of simulations by trial-and-error and averaging procedures with the criteria set by P₂ values (further discussion below). The COL monomer numbers are selected to elucidate the number effects in a qualitative manner.

### P₂ Parameter

The P₂ parameter is extensively used in previous works in characterizing the alignment of a single chain polymer or bulk polymers. For example, in the work of Lin et al., this parameter is used to effectively track the alignment of bulk polyethylene under thermal drawing. The parameter is describing the polymer chain conformation change as a function of strains in the work of Liu et al., which finds the correlation between P₂ and enhanced thermal conductivity; furthermore, the uniaxially stretched poly(vinylphenol) is also effectively analyzed with this parameter, which successfully characterizes the changes of the orientation of the main chain, benzene segments, and hydrogen bonds. In summary, P₂ is a qualitative measure that fluctuates between perfectly aligned (value of 1) and entirely disordered (value of 0); a greater number generally indicates greater alignment. The P₂ measure described herein is increasing over time until it plateaus, e.g., becoming more aligned over time.

### Fibronectin Fluorescent Labeling

Fibronectin (FN) from human plasma was fluorescently labeled with Alexa Fluor 488 TFP (A488) according to previous work. The pH of the protein solution was adjusted to pH 9.0, and a 15:1 molar ratio of A488 to FN was added and gently aspirated to mix the two components. The solution was left at room temperature for one hour to give the fluorophores adequate time to bind, and excess fluorophore was removed from the solution. The fluorescent analysis and labeling efficiency were determined according to the method produced by Paten et al.

Incorporation of Fibronectin into the Collagen Scaffold

A collagen (COL) scaffold was produced by preparing a neutralized 2.5 mg mL⁻¹ COL solution and spinning it in the device at 75 s⁻¹. This process was repeated three more times to produce a four layered scaffold. For the fifth layer, a 0.5 mg mL⁻¹ neutralized COL solution was made with the addition of fluorescent FN at a 1:5 molar ratio of fluorescent FN to COL; this solution was spun at 825 s⁻¹ to adjust for the lower solution viscosity. The scaffold was then removed from the spindle in the standard manner and transferred to a Delta-T dish (Bioptechs, 04200417C) filled with 1×PBS, and rinsed 10 times by removing half of the solution and replacing it with fresh 1×PBS to remove any unbound fluorescent FN. The network was then imaged with an inverted microscope equipped with a CMOS camera (Andor, Zyla 4.2). For fluorescent imaging, the excitation signal from the fluorescent FN went through a 470±20 nm filter, and the emission signal passed through a 525±25 nm (Chroma Technology, 49002) before the signal was captured. To determine whether the FN that was incorporated into the network was in a fibrillar or globular form, a 4% (v/v) sodium deoxycholate was prepared in 1×PBS solution, and half of the solution in the Delta-T dish was replaced with the sodium deoxycholate for a final concentration of 2% (v/v) and left to sit at room temperature for one hour. The solution was then rinsed to remove the sodium deoxycholate from the solution and then scaffold was imaged again.

### Fibril Alignment Analysis

Each SEM image was analyzed via a MATLAB code designed by Sander et al that measures the fibril distribution in an image (Sander, E. A.; Barocas, V. H., Comparison of 2D fiber network orientation measurement methods. J Biomed Mater Res A 2009, 88 (2), 322-31). Before analysis, the SEM image was first cropped into a square for the most accurate analysis. Then, the Fourier Transform filters were adjusted: In summary, the upper Fourier Transform filter in the MATLAB code was adjusted by measuring the average fibril diameter (10 measurements) in the image, doubling the value, and converting it to a frequency value based on the dimensions of the image (Equation 1). This value was then increased by 10% to improve the accuracy of the analysis and adjusted to a filter value unique for the code (Equation 2). The lower Fourier Transform filter was held constant due to a negligible effect on the overall results.

$$Frequency = \frac{Pixel Width of Image}{( 2 \times Average Fibril Diameter )} \qquad ( 1 )$$

$$Filter = \frac{Adjusted Frequency}{Pixel Width of Image} \qquad ( 2 )$$

For the analysis, the code first converts the square image to the frequency domain with the fast Fourier Transform method, and then into polar coordinates. The program then determines the number of points that contribute to each angle between 0-360° and measures the frequency of each angle in the image. The orientation tensor was also calculated for each image. After the code is complete, it was determined the range of angles needed to include 68% (or one standard deviation) of the fibrils. For each trial, five images were analyzed, and the average fibril range was determined. Each condition consists of three trials.

### Computational Methods:

Molecular dynamics (MD) simulations can capture changes in material properties over time at the molecular scale. This unique feature paves the way towards examining dynamic material phenomena, such as molecular self-assembly, at time and length scales that are beyond the reach of conventional experimental approaches (Lin, S.; Cai, Z.; Wang, Y.; Zhao, L.; Zhai, C., Tailored morphology and highly enhanced phonon transport in polymer fibers: a multiscale computational framework. npj Computational Materials 2019, 5 (1), 1-12; Zhai, C.; Zhou, H.; Gao, T.; Zhao, L.; Lin, S., Electrostatically tuned microdomain morphology and phase-dependent ion transport anisotropy in single-ion conducting block copolyelectrolytes. Macromolecules 2018, 51 (12), 4471-4483; Zhai, C.; Li, T.; Shi, H.; Yeo, J., Discovery and design of soft polymeric bio-inspired materials with multiscale simulations and artificial intelligence. Journal of Materials Chemistry B 2020; Wang, M.; Feng, Z.; Zhai, C.; Zhou, Q.; Wei, T.; Liu, J., Chromium carbide micro-whiskers: Preparation and strengthening effects in extreme conditions with experiments and molecular dynamics simulations. Journal of Solid State Chemistry 2020, 291, 121598; Zhai, C. Understanding Slow Dynamics in Polymers Using Coarse-Grained Molecular Dynamics Simulations: Microphase Separation in Block Copolyelectrolytes and Photo-Switching in Azobenzene-Polymers. The Florida State University, 2020). For instance, all-atomistic (AA) and coarse-grained (CG) molecular modeling and simulations were instrumental in making large strides in fundamentally understanding the mechanical behavior of collagen fibrils (Depalle, B.; Qin, Z.; Shefelbine, S. J.; Buehler, M. J., Influence of cross-link structure, density and mechanical properties in the mesoscale deformation mechanisms of collagen fibrils. Journal of the mechanical behavior of biomedical materials 2015, 52, 1-13; Buehler, M. J., Atomistic and continuum modeling of mechanical properties of collagen: elasticity, fracture, and self-assembly. Journal of Materials Research 2006, 21 (8), 1947-1961; Gautieri, A.; Russo, A.; Vesentini, S.; Redaelli, A.; Buehler, M. J., Coarse-grained model of collagen molecules using an extended MARTINI force field. Journal of Chemical Theory and Computation 2010, 6 (4), 1210-1218; Zhang, N.; Cheng, Y.; Hu, X.; Yeo, J., Toward rational algorithmic design of collagen-based biomaterials through multiscale computational modeling. Current Opinion in Chemical Engineering 2019, 24, 79-87). Therefore, an extensively validated CG collagen model was used to describe the spinning behavior of the collagen molecules, as this CG model could accurately capture the mechanical behavior of individual collagen molecules as well as collagen fiber bundles.

A mesoscopic CG model consisting of beads of 1-1.5 amino-acids, which correspond to 10-20 atoms, was developed. This CG model is derived from a high-fidelity AA triple helical tropocollagen model to enable simulations at larger time and length scales. The initial configuration of the beads was spaced at 14 Å at equilibrium, which is close to the diameter of a collagen molecule. The simulation box was set to be longer than the collagen molecule to avoid interactions between opposing ends of the same molecular chain due to the periodic boundary conditions.

The paired, bonded, and angular potentials that contribute to the total energy of the interacting beads in the CG model was described with a Lennard-Jones (11) style interaction:

$${E}_{paired} = 4 {ε}_{LJ} [ {( \frac{{σ}_{LJ}}{r} )}^{12} - {( \frac{{σ}_{LJ}}{r} )}^{6} ] \qquad ( 3 )$$

where ε_{LJ}=11.06 Kcal/mol is the dispersion energy and σ_{LJ}=14.72 Å is the equilibrium distance between two interacting beads where the LJ potential energy was zero. The bonded interaction in this model was described as:

$${E}_{bonded} = \{ \begin{matrix}{{k}_{1} ( r - {r}_{0} )}^{2} , & r < {r}_{1} \\ {{k}_{2} ( r - {r}_{0} )}^{2} , & {r}_{break} > r > {r}_{1} \\ 0 , & r > {r}_{break}\end{matrix} \qquad ( 4 )$$

where k₁=17.13 kcal/mol/Å² and k₂=97.66 kcal/mol/Å² are the spring constants in small and large strains, r₀=14 Å is the equilibrium distance of the spring, r₁=18.2 Å is the critical hyper-elastic distance, and r_{break}=21 Å is the bond breaking distance. The angular potential was calculated as:

E_{angular}=k_{angular}[β−β₀]² (5)

where k_{angular}=14.98 kcal/mol/rad² is the bending stiffness and β₀ is the equilibrium angles that range between 170°-180°.

The local chain alignment at each bead i can be described using a vector

$${e}_{i} = \frac{{r}_{i + 1} - {r}_{i - 1}}{\vert {r}_{i + 1} - {r}_{i - 1} \vert} .$$

Therefore, the alignment parameter, P₂, was used to describe the orientation along the shearing direction:

$${P}_{2} = \frac{{\sum }_{i = 1}^{N} 3 〈 {( {e}_{i} \cdot {e}_{shear} )}^{2} 〉 - 1}{2 N} \qquad ( 6 )$$

where eₛₕₑₐᵣ is the unit vector along the shear direction and N is the total number of beads. When P₂ was above a threshold value of 0.6, the collagen molecules were categorized as mostly aligned in the corresponding shearing direction.

The collagen molecules were equilibrated for 2 ns before the shear and the shear simulations were ran for enough time until P₂ reached above 0.6 (collagen molecules fully aligned) or fluctuated around a certain value below 0.6 (would not align eventually). All the simulations were performed using the NVT ensemble.

### Cross-Section SEM Imaging

To measure the thickness of the scaffold, the buffer solution was slowly replaced with 95% ethanol solution; half of the buffer volume was removed, and the same volume was replaced with ethanol. This process was repeated eight times with a 10-minute delay between each cycle. Afterwards, a thin glass coverslip was placed into the solution and the scaffold was carefully positioned on top of it while both remained submerged in ethanol. The ethanol was then left to evaporate in a fume hood, which caused the COL network to dry onto the glass coverslip. The coverslip with the scaffold was then submerged in liquid nitrogen and fractured lengthwise down the scaffold. The pieces were then removed and attached to the side of a SEM stub with carbon tape so the cross-section of the scaffold could be imaged. The samples were sputter coated with 2.5 nm of platinum before SEM analysis. Three scaffolds were prepared for each condition (three, five, and seven layers), and five images were taken of each scaffold.

### Preparing Scaffold for Thickness Measurements

To prepare the scaffolds for thickness measurements, the samples were cross-linked using a 2.5% (v/v) glutaraldehyde (in 1×PBS) and then buffer-exchanged by replacing half of the solution volume with 1×PBS solution seven times. They were then dehydrated in 95% ethanol through a graded series of solution exchanges. A glass coverslip was placed in the ethanol under the scaffold, and the ethanol was then left to evaporate in a fume hood, which caused the material to dry onto the glass coverslip. The coverslip with the scaffold was then submerged in liquid nitrogen and fractured lengthwise down the scaffold. The pieces were sputter coated with 2.5 nm of platinum before SEM imaging. Three scaffolds were prepared for each condition (three, five, and seven layers), and five images were taken of each scaffold.

### Scaffold Thickness Analysis

The thickness of the scaffolds was determined from the cross-section SEM images using ImageJ. Fifteen measurements were taken for each independent sample across five fields of view. Each condition consisted of three independent samples. The average thickness and the standard deviations of each condition were calculated from these measurements.

### Scaffold Thickness Analysis

The thickness of the scaffolds was determined from the cross-section SEM images using ImageJ. Five measurements were taken for each image and five images were taken for each trial for the three conditions (three, five, and seven layers). Each condition consisted of three trials. The average thickness and the standard deviations of each condition were calculated from these measurements.

### Mechanical Testing

For hydrated scaffold tests, a 5-layer scaffold (made with 2.5 mg/mL COL and spun at 75 s⁻¹) was prepared and then removed from the buffer solution by gripping a shorter end of the scaffold with tweezers and slowly removing it from the buffer so it folded in on itself. The folded scaffold was then placed in a custom-built mechanical testing device attached to a high-resolution linear actuator with a DC stepper motor controlled by a MTESTQuattro controller (ADMET)and secured with custom-built stainless-steel grips. To prevent slipping, 2 μL of tissue glue (3M Vetbond) was added to the grips right next to the clamped scaffold and left to dry for ten minutes. The mechanical test chamber was then sealed and 12 mL of 1×PBS buffer was added to the chamber to immerse the scaffold. The scaffold was left to hydrate for ten minutes. The scaffold was then pulled at 0.05 mm/sec until it was under 0.005 N of load; this marked the point when the scaffold had no slack left. The scaffold was then pulled at 0.1 mm/sec until breakage. The process was repeated with two more freshly made scaffolds for an n=3.

For the dried scaffold tests, a scaffold was prepared with the same parameters and removed from the buffer solution in the same manner. This time, the scaffold was laid on a piece of parafilm and left to dry in a vacuum for one hour. The same protocol used for securing and pulling the hydrated scaffolds were used for the dried ones as well. The process was repeated with two more new scaffolds for an n=3.

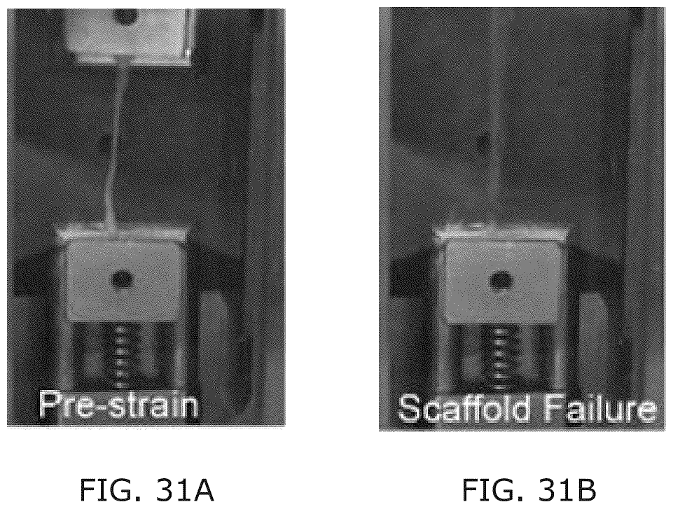

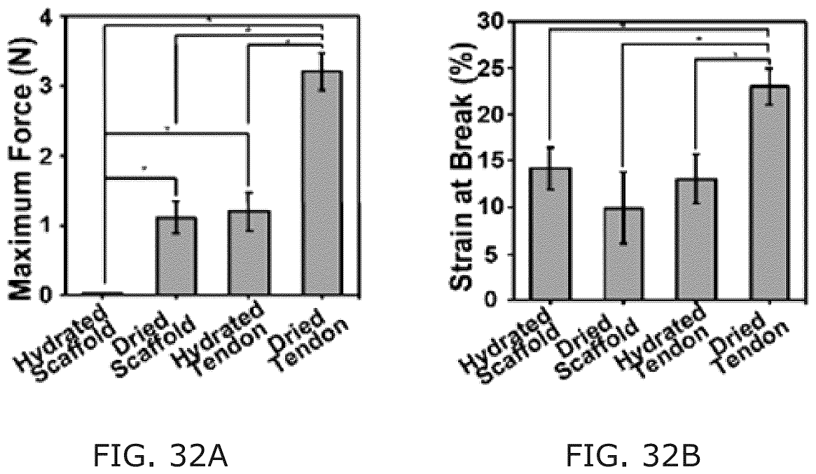

For the analysis, the reported force values in FIGS. 31A-B and FIGS. 32A-B are the maximum force the scaffold endured before breakage. The maximum strain was calculated as the difference between the scaffold length at break and the initial scaffold length divided by the initial scaffold length.

### Statistical Analysis

Statistical analysis was completed in Microsoft Excel with a one-way ANOVA test where the p-value equals 0.05. The null hypothesis stated that there is no significant difference between the tested conditions. In the analysis, if F>F_{crit} then at least one condition was different from the other conditions and the null hypothesis was rejected. To determine which conditions were different from each other, a Tukey test was performed where the p-value was equal to 0.05.

### Results

### Formation of Anisotropic COL Scaffolds

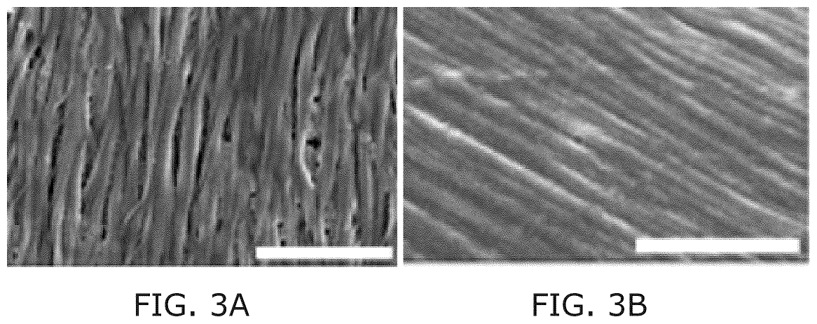

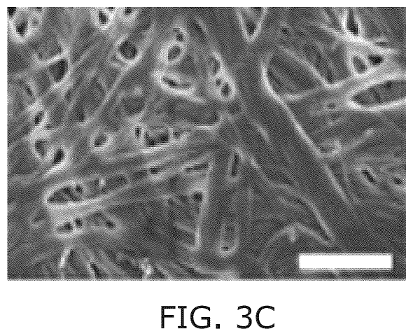

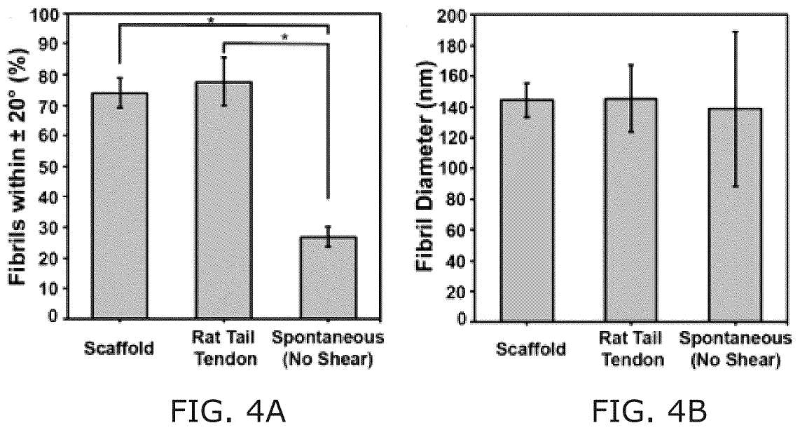

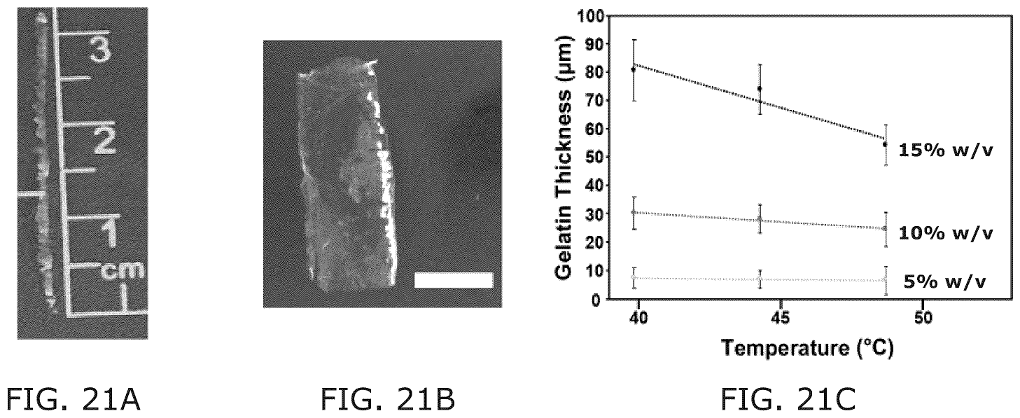

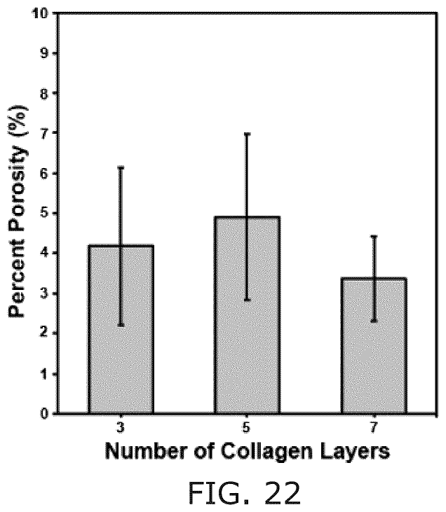

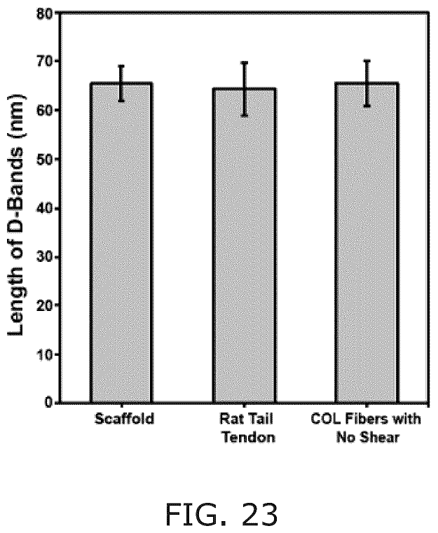

To generate sheets of COL fibrils, a device with two concentric cylinders, where the inner cylinder (the spindle) rotated, and the outer cylinder (the reservoir) remained stationary (FIG. 1A); the gap space between the two cylinders remained constant at ˜1 mm was designed. This setup allowed for neutralized COL to spontaneously polymerize under shear flow, leading to the formation of a fibrillar scaffold (FIG. 2A). Prior to spinning, the spindle was coated with a uniform layer of un-crosslinked gelatin with a thickness of 28.3±5.0 μm, where the gelatin acts as a surface for the collagen to adhere, or tether, to during fibrillogenesis (FIGS. 21A-C). Without the gelatin layer, polymerized COL formed only as a thin strip at the air-liquid interface (FIG. 21A); furthermore, if no shear is present during fibrillogenesis, then no fibrils form on the gelatin-coated spindle. These observations point to a synergistic relationship between the assembling COL solution and the gelatin interface, which it is believe to be required to initiate adhesion of monomers, nucleation of fibrils, and ultimate generation of a continuous fibrous material. In the presence of the gelatin, COL fibrils spanned the entire spindle length, enabling their collection as a ∞3.0×1.0 cm scaffold with a porosity of only 4.9±2.1%, highlighting the tightly packed nature of the fibrils (FIG. 2B, FIG. 21B, and FIG. 22). Perhaps more importantly, the gelatin served as a sacrificial layer that allowed for an easy removal of the scaffold from the spindle without disrupting the anisotropic substructure. The presence of native D-banding on the COL fibrils indicated that the shear-induced alignment did not disrupt the natural polymerization of COL (FIG. 2C); the bands had an average width of 65.5±3.6 nm, which is comparable to band widths published in the literature, and the D-banding that was imaged on a rat tail tendon (FIG. 23). Furthermore, the exquisite fibril alignment, uniform D-banding, and consistent fibril diameters that persisted throughout the scaffolds were comparable to those found in isolated rat tail tendons, but the fibril alignment in the scaffolds differed significantly from fibrils formed in a COL gel in the absence of shear (FIG. 3A-C and FIG. 23). It was calculated that 74.0±3.2% of the shear-aligned fibrils were within ±20° of the orientation of all fibrils in the scaffold. This alignment was statistically similar to the 77.7±7.9% alignment calculated from fibrils in an isolated rat tail tendon specimen and significantly differed from the scaffold formed with no shear (FIG. 4A). Despite how they were made, fibril diameters remained consistent in each sample, indicating that the shear flow from the device did not inhibit the natural polymerization process (FIG. 4B).

### Analysis of the Mechanism Behind Fibril Alignment

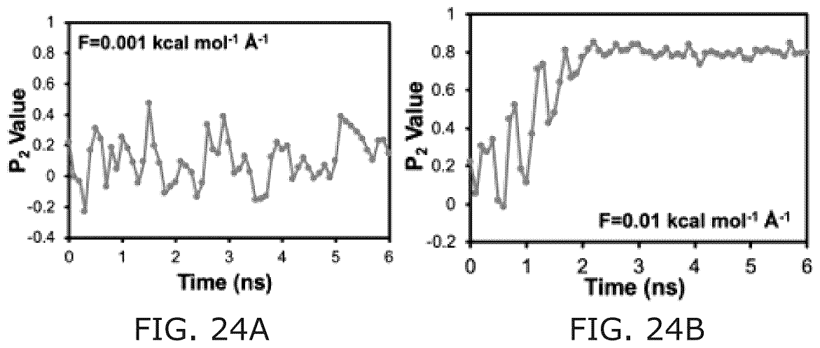

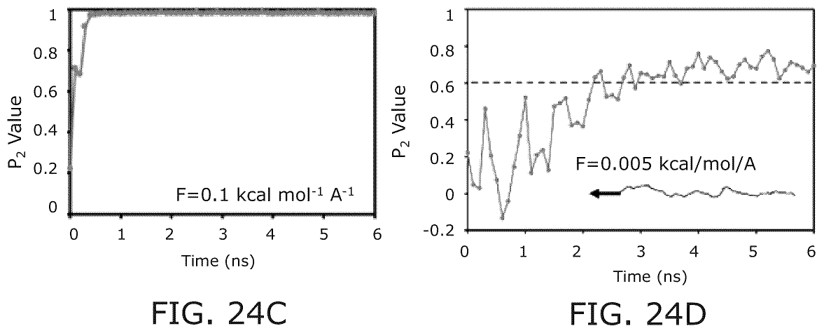

After examining the effects of both shear and the presence of the gelatin layer on the formation and alignment of COL fibrils, the next goal was to look at the effect of these variables on individual COL molecules in parallel to the full fibers. The goal was to gain insight into the mechanism behind the COL fibril alignment that is observed by examining the initial effect that shear flow had on the COL monomers before fibril formation. To further examine the impact of tethering on the dynamic alignment of COL molecules under shear, a CG simulation framework was constructed to qualitatively simulate molecules as beads, where one bead experienced a tethering force at the end of the chain. Here, tethering force was defined as the anchoring force that keeps one end of the molecule stationary, such that the molecule only experiences application of a uniaxial force. In this configuration, the simulations emulated the initial deformation of the COL molecules experiencing this tethering force, which was defined as the uncoiling and alignment of the molecules on the spindle. To determine if the molecules aligned in the direction of the applied force, the uniaxial order parameter, or P₂, value in the system was calculated. Under these conditions, it was observed that a single COL molecule gradually aligned within approximately 3 ns when the magnitude of the tethering force was above 0.005 kcal/mol/Å (FIG. 5A). When the tethering force was below this threshold, the simulated COL did not overcome the pairwise, long-range cohesive/attractive forces between two interacting beads and resulted in an unaligned structure (FIGS. 24A-D). Above this threshold, the tethering force could surmount these opposing forces and initiate the uncoiling of the COL molecule, which eventually facilitated overall alignment of the structure.

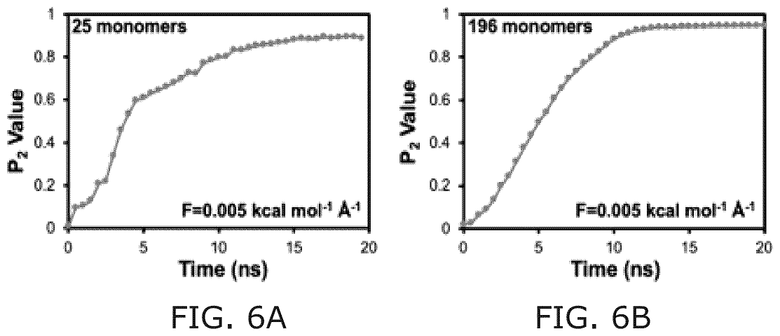

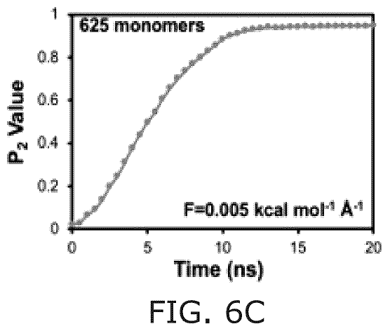

To expand on these findings, it was next calculated the threshold force required to align COL chains that were 1-, 5-, and 10-monomers long. It was observed an increase in threshold force as the chain length increased likely due to the larger intramolecular adhesion and pairwise attractive forces that intensified as the number of CG beads increased (FIGS. 5A-C). Interestingly, it was also found that the minimal force needed to align multiple monomers of COL was the same as that of a single COL molecule, suggesting that the starting COL concentration should not significantly impact alignment (FIGS. 6A-C). The intermolecular interactions experienced between multiple chains of the shorter, monomeric COL molecules during shear were beneficial for catalyzing alignment within the system.

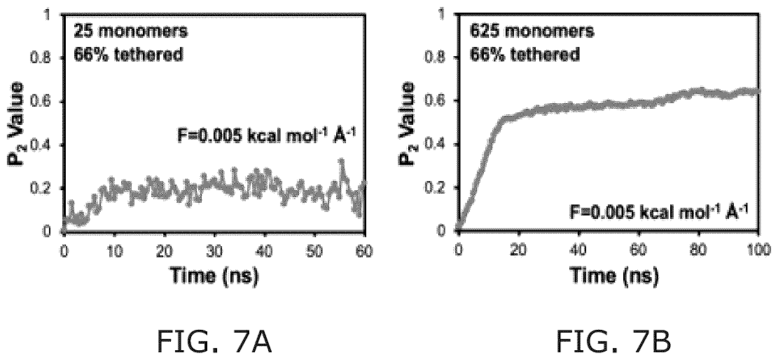

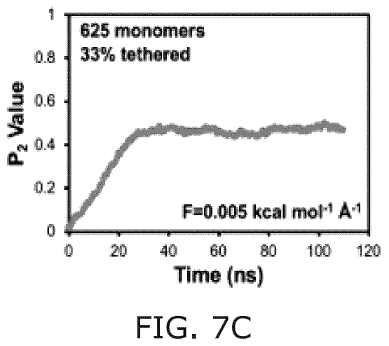

The role of molecular tethering on the overall alignment of the multi-chained systems was explored. In these studies, the percentage of molecules was varied that experienced tethering and monitored how these changes impacted the P₂ value (FIGS. 7A-C). In a system with only 25 COL chains, alignment was worse when 66% of the chains were tethered, as indicated by the low P₂ value (FIG. 7A). However, it was found that overall alignment increased in a 625-chain system when either 100% (FIG. 6C) or 66% (FIG. 7B) of all chains were tethered, and misalignment reigned when only 33% of the chains were tethered (FIG. 7C). This difference indicated that tethering was necessary for overcoming entropic effects and inducing system-wide alignment within starting solutions consisting of concentrated monomers (e.g., more available chains of shorter COL molecules). Based on these findings, it was deduced that a portion of the molecules needed to be tethered to drive scaffold formation and ultimate alignment.

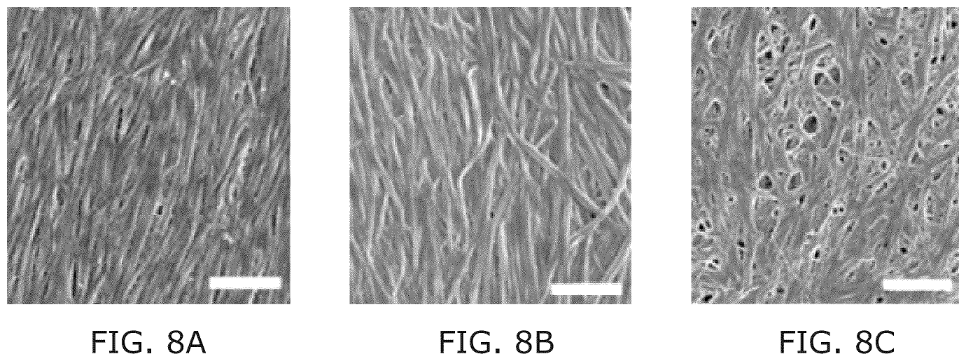

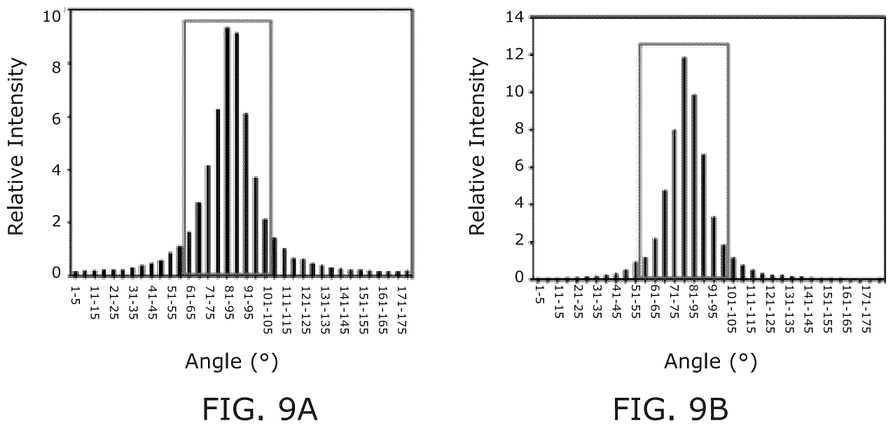

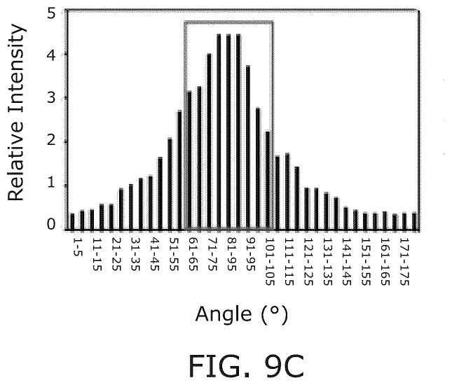

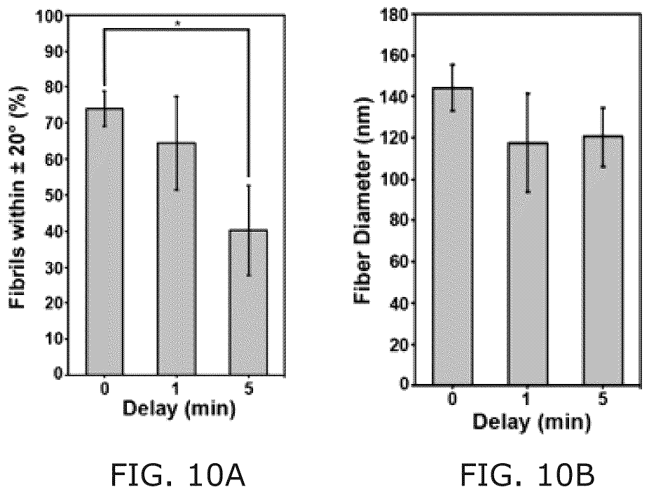

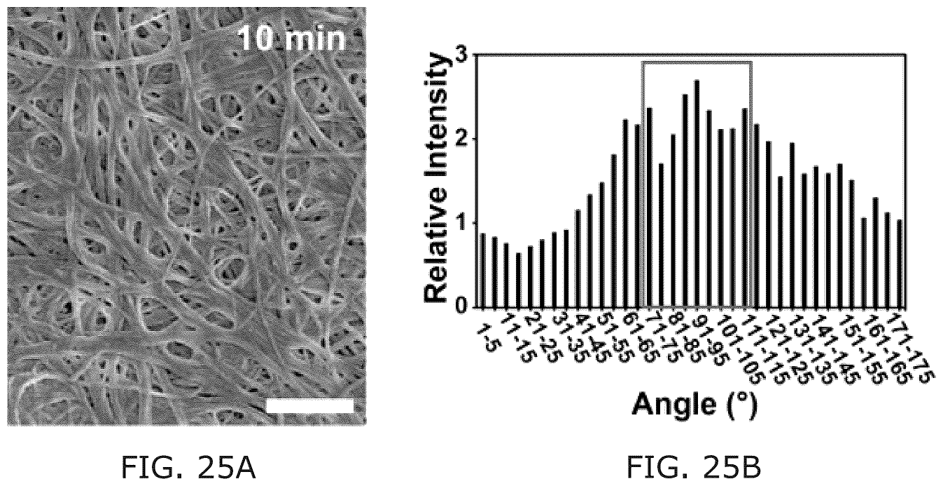

To expand on these results, the effects of pre-polymerization (indicative of a growing COL chain) on alignment (FIGS. 8A-C and FIGS. 9A-C) were monitored. In these studies, the spinning process was delayed for 1 and 5 minutes after triggering COL polymerization and compared changes to alignment with a solution that was simultaneously polymerized under shear (e.g., 0-minute delay). Fibril alignment in the scaffold decreased from 74.0±4.9% to 64.4±13.0% to 40.2±12.5% at a 0-, 1-, and 5-minute spin delay, respectively (FIG. 10A). These results suggest that the COL fibrils had grown long enough during the delay period, where shear flow was no longer capable of aligning the structure. In each case, there were also no notable changes to fibril diameter (FIG. 10B). When the COL was left to polymerize for 10 minutes before spinning was engaged, the fibrils were unable to adhere to the gelatin coating; instead, they formed a bundle of isotropic fibers at the bottom of the reservoir (FIGS. 25A-B). These results were in agreement with the computational predictions, supporting that the shear force in the system was required to trigger adhesion between the spindle and COL monomers and that partial pre-polymerization under no shear decreased the number of tethered COL molecules and fibril alignment. When taken together, the computational and experimental findings suggested that a combination of larger critical forces and longer spinning times would be necessary to align the pre-polymerized COL fibers. In summary, both the gelatin tethering layer and simultaneous spinning/polymerization of COL were needed to achieve eventual alignment; without these design features, the resultant material became disordered.

### Tuning Shear Rate to Maintain Fibril Alignment

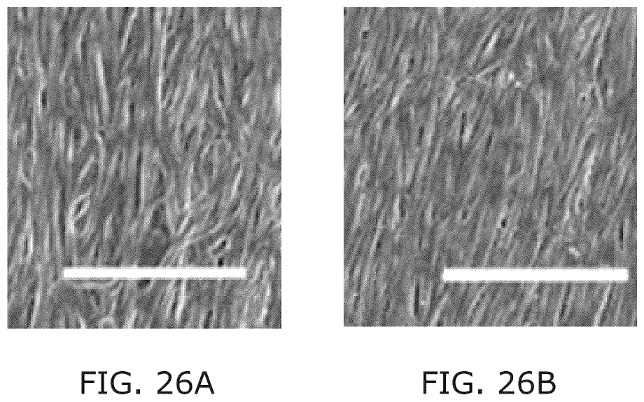

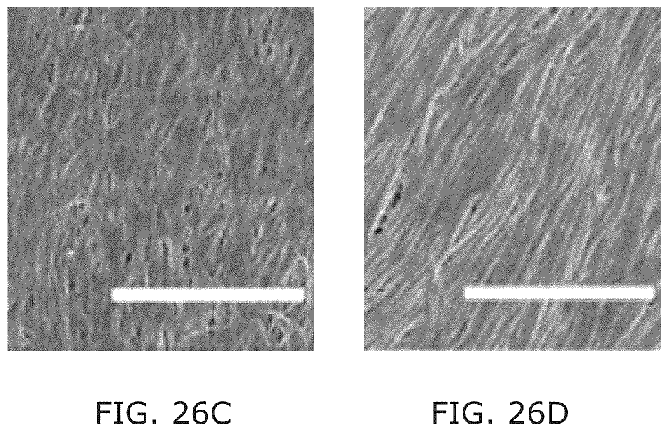

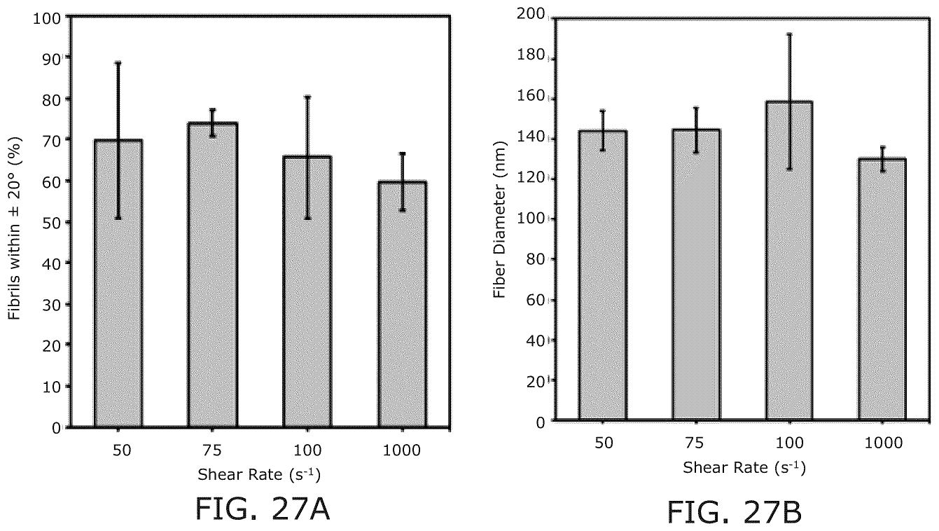

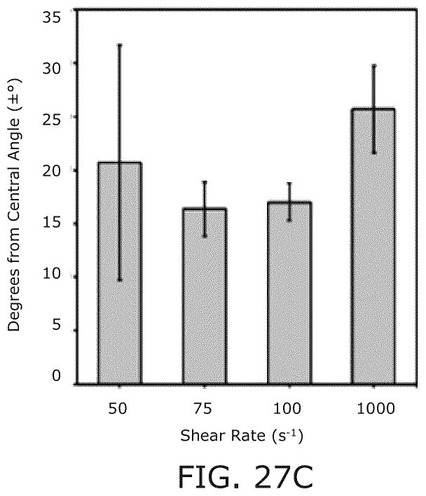

Given the dependence on spin time and tethering capacity on the alignment and growth of the scaffolds, other variables regulating scaffold formation were considered. Shear rates of 50, 75, and 100 s⁻¹ were first investigated, where it was found 69.7±18.9%, 74.0±3.2%, and 65.5±14.8% of fibrils fell within ±20° of the orientation tensor respectively, and that the shear rate had no influence of fibril diameter (FIGS. 26A-D and FIG. 27A-C). To establish the maximum shear rate that could achieve without losing alignment, scaffolds at 1000 s⁻¹ were prepared and found that the alignment was not significantly different from that in other scaffolds (FIG. 26D and FIG. 27A). This is not surprising, as the calculated Reynolds numbers suggested that the flow produced by the rotating spindle at these shear rates was laminar (Table 1).

TABLE 1
Reynold's Number in Concentric Cylinder Device

| Shear Rate (s⁻¹) | Reynold's Number |
|---|---|
| 50 | 1.5 |
| 75 | 2.3 |
| 100 | 3.0 |
| 1000 | 21.9 |

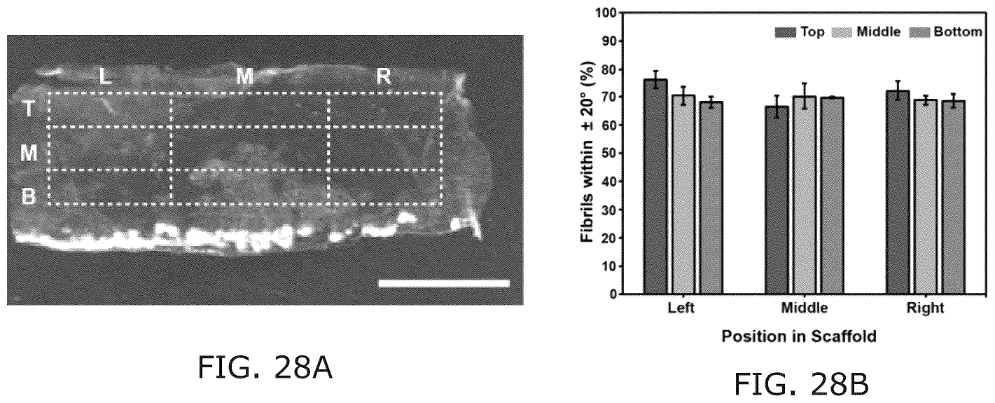

At shear rates above 1000 s⁻¹, the gelatin coating was torn off the spindle, preventing scaffold formation altogether. Even though the results were not significantly different from each other, fibril organization was greatest at 75 s⁻¹ and was sufficient to induce broad area alignment across the entire scaffold (FIGS. 28A-B).

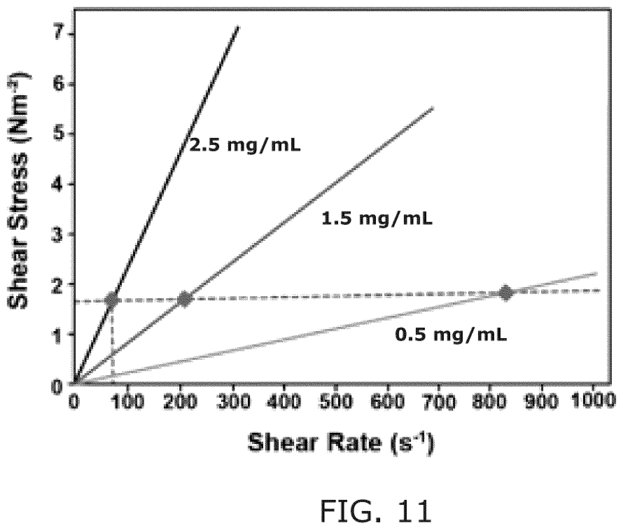

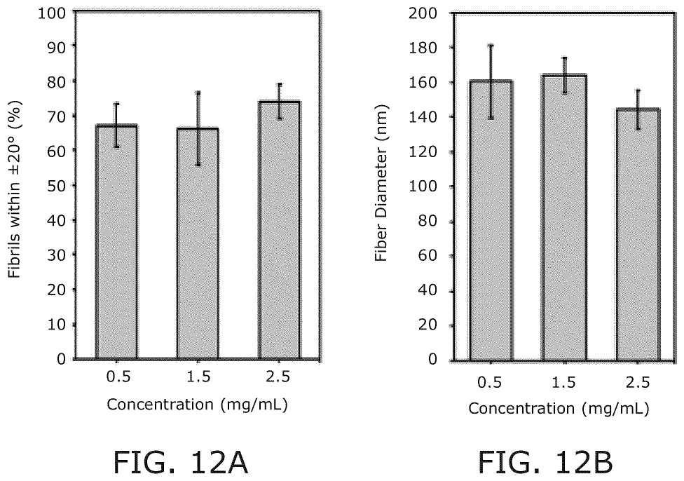

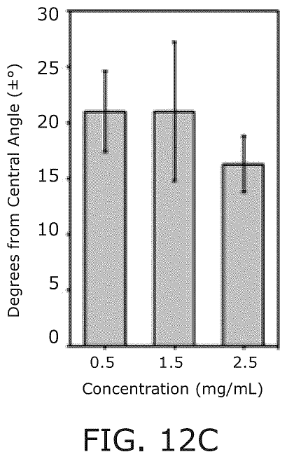

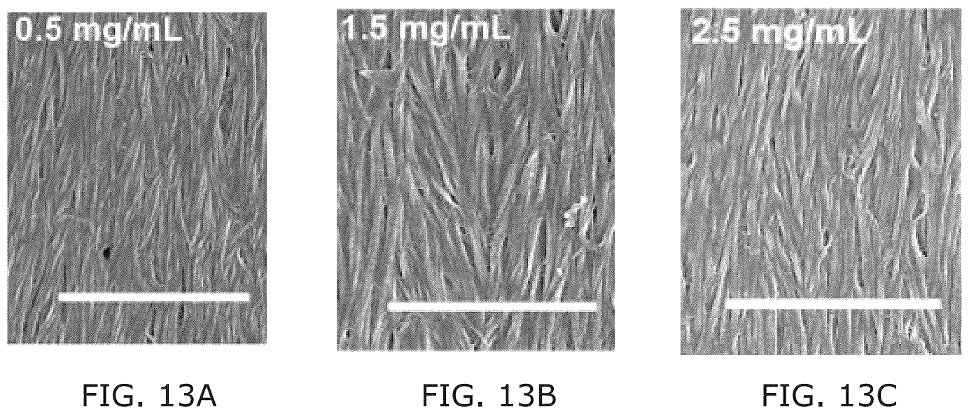

Negligible changes to fibril alignment as a function of shear rate were observed. Maintaining the shear stress of the system to preserve fibril alignment at multiple concentrations of COL was studied. Because shear stress is directly related to the shear rate and viscosity, it was approximated that a constant shear stress of 1.7 N/m² (the amount of shear the system applies at 75 s⁻¹) could be maintained by adjusting the rotational speed for solutions prepared at different concentrations of COL (FIG. 11). When shear stress was maintained, statistically similar alignment for each condition was achieved (67.1±6.1%, 66.1±10.4%, and 74.0±3.2% for 0.5, 1.5, and 2.5 mg/mL, respectively (FIGS. 12A-B). These findings supported that shear stress was critical to maintaining fibril alignment (FIG. 12A) and fibril diameter (FIG. 12B) even when other parameters (i.e., shear rate and concentration) were altered.

### Increasing Scaffold Dimensions and Composition

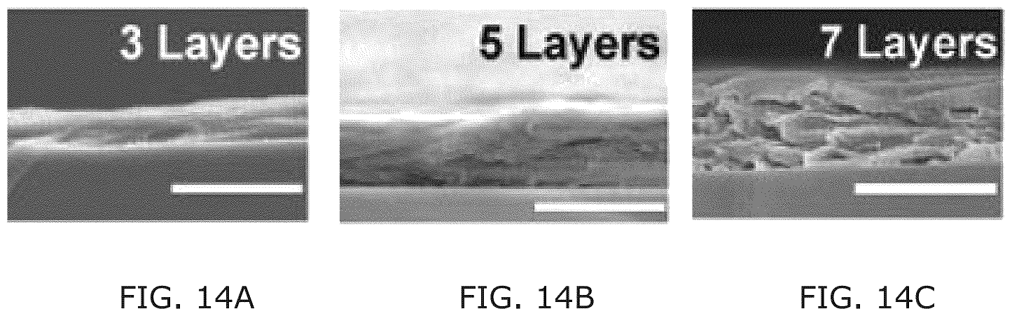

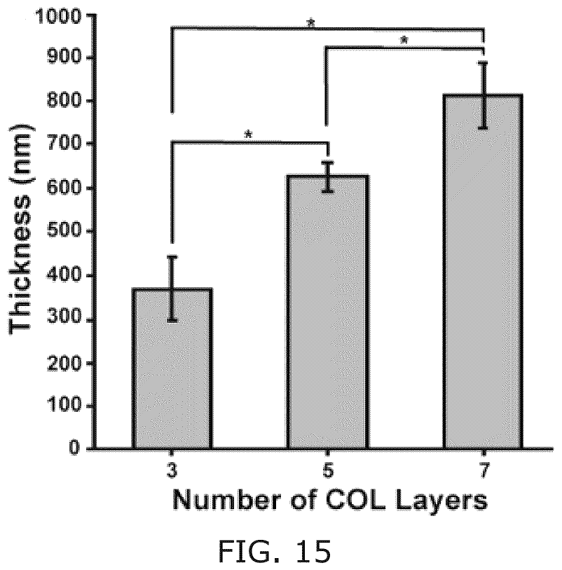

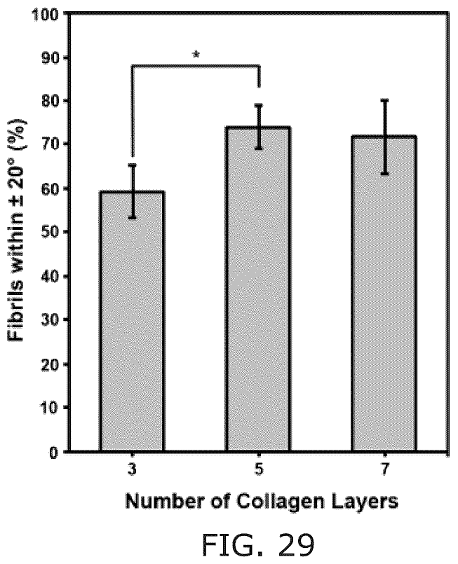

To investigate the tunability of this platform, it was next varied the dimensions (length, width, and thickness) of the scaffold and measured the impact on fibril alignment. The number of COL layers were changed in the scaffold and found that the thickness of dried 3-, 5-, and 7- layered scaffolds (e.g., repetitive spin cycles) increased to 369±73 nm, 625±32 nm, and 813±76 nm, respectively (FIGS. 14A-C and FIG. 15). It was interesting to note though that when the shear rate was held constant, fibril alignment increased when the number of COL layers increased from 3 to 5, suggesting that previous layers partially templated the alignment of new fibrils (FIG. 29). However, there was no significant improvement in fibril alignment between the 5- and 7- layered scaffolds, indicating that the fibril organization is maintained even when the thickness is further increased. Finally, it was noted the porosity of the 3- and 7-layered scaffolds remained statistically similar to the previously mentioned porosity of the 5-layered scaffold at 4.1±2.0%, 4.9±2.1%, and 3.4±1.1% for the 3-, 5-, and 7-layered scaffolds respectively (FIG. 22).

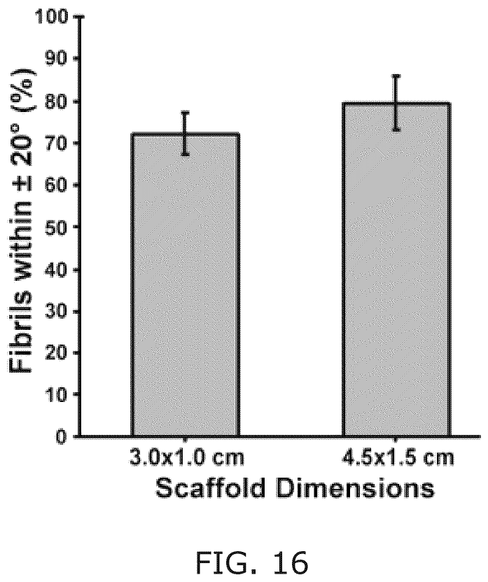

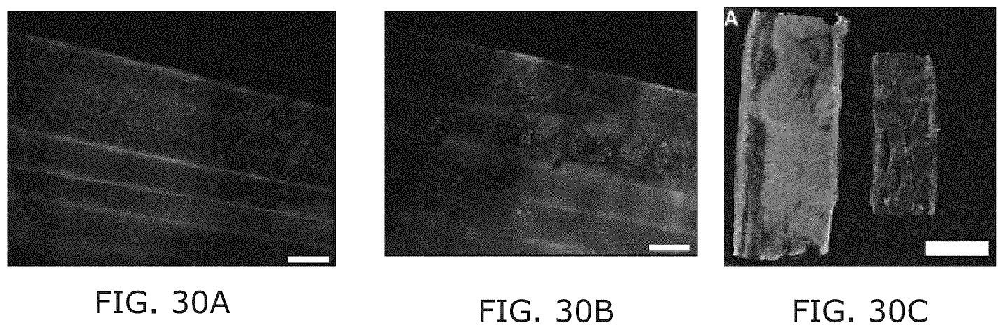

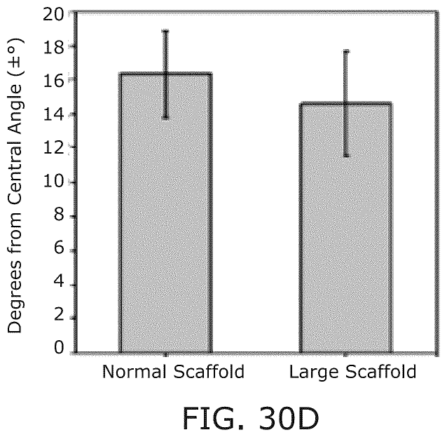

To complement the results from the thickness analysis, the increase in the length and width of the scaffolds impacted structure formation and ultimate alignment was studied. To test this, the footprint of the device was increased by 50% in a new design which, as a result, increased the size of the scaffold to approximately 4.5×1.5 cm. This increase in size did not significantly alter the fiber alignment throughout the scaffold when compared to the smaller 3.0×1.0 cm scaffold, indicating that scaling this design was feasible without sacrificing the underlying substructure of the scaffolds (FIG. 16). Finally, it was tested whether other fibrillar proteins could be incorporated to diversify the scaffold composition. To test this final variable, a soluble fibronectin (FN) was blended into the starting COL solution and prepared the scaffolds. It was observed the presence of fibrillar FN that persisted throughout the scaffolds. To confirm that the FN was indeed in its fibrillar form, the scaffold was incubated in 2% sodium deoxycholate, a chemical that digests globular but not fibrillar FN and observed a FN positive fluorescence signal that indicated its presence in the fibrillar form in the scaffold (FIGS. 30A-B). When taken together, these findings suggested that the system can be tuned to enable the production of multi-component and multi-dimensioned scaffolds—both are important characteristics when considering designs for future tissue engineering applications.

### Discussion

A device was designed, built, and tested that leverages Couette-flow to generate shear and align COL, as it spontaneously polymerizes under ambient conditions. While the use of shear to align COL fibrils is not new, this approach is among the first to incorporate fluid flow together with dynamic polymerization to align and grow COL scaffolds with anisotropic substructures. The optimal shear rate of 75 s⁻¹ is comparable to other reports that have generated aligned COL fibrils including Saeidi et al. (20 and 80 s⁻¹), Nerger et al. (100 s⁻¹), and Lai et al. (˜200 s⁻¹). Unlike previous work, it was validated that proper COL polymerization has occurred through the presence of D-banding along each of the fibrils, where the banding was comparable to those in the rat tail tendons and from native COL fibrils reported in the literature (FIG. 23). Because this process can simultaneously nucleate, tether, and grow COL fibrils during scaffold formation, these signature anisotropic features with negligible surface defects are retained across the centimeter scale of the materials.

The incorporation of the sacrificial gelatin layer during scaffold formation is another differentiating but essential feature of the platform that not only improved the dimensions and practical handling of the material, but also contributed to fibril alignment during polymerization. COL monomers interact with each other via noncovalent interactions, and it was suspected that electrostatic and hydrogen bonding occurs during polymerization; since gelatin is simply denatured COL, it is likely that similar interactions occur during scaffold development. Despite this natural interaction, scaffold formation on the spindle only occurs under shear flow. These findings were further supported by computational analysis, which suggested that once the molecular alignment process was initiated under flow, it facilitated the uncoiling of neighboring, unaligned molecules via intermolecular repulsion as a result of limited wiggle room. This hypothesis was supported by the steep increase in P₂ in FIG. 7B, which denoted the structural evolution of the tethered molecules, followed by its gradual increase over time attributed to an increased susceptibility of untethered molecules to align in the presence of the tethered chains. Because the single tropocollagen molecule was simulated with a concentration calculated to be 0.3×10⁻⁴ mg/mL, which is about four orders of magnitude more dilute than the most dilute experimental COL solution (0.5 mg/mL), only the very initial stages of the aligning process were simulated to provide some fundamental, qualitative insight comparing to the experimental large scale of micro- to milli-meters. When taken together with experimental data, these findings highlighted that molecular tethering is a requirement for alignment. When the start time for spinning the COL was delayed—indicative of a conversion of molecules to fibrils—a lower percentage of molecules could be tethered, making alignment inconsistent and, in some cases, impossible.

The exploration and characterization of the planar fibril structures produced in this report were necessary for understanding the molecular mechanisms underlying scaffold formation; however, the true translatable features of the approach lie its design versatility and compositional customizability—two attributes that were demonstrate in a proof-of-concept approach in FIGS. 14A-C and FIG. 15. Because the spindle and outer chamber are CAD-based designs, they can be easily reconfigured and scaled to generate multiple shapes and structure. It was demonstrated that two dimensions of a cylinder, but the design could conceivably be reconfigured to multiple geometries and structures. By systematically increasing the number of spin cycles, the thickness of the scaffold can be increased while maintaining fibril anisotropy, which has shown to be a critical step in generating a usable material. If the spin cycle is not repeated it is difficult to both remove and handle the resulting scaffold without causing significant damage. However, performing multiple spin cycles not only adds to the durability of the material, but also contributes to fibril alignment (FIG. 29). Other researchers have utilized aligned materials, such as dextran fibers and micropatterned glass slides to align COL fibrils during the polymerization process. It is possible that in the system the already polymerized fibers act as a template for polymerizing fibrils and may 1) serve as nucleation sites for further layers, or 2) display stronger electrostatic and hydrogen-bonding interactions with the polymerizing COL than the smooth gelatin layer does. Overall, the process of repeating spin cycles is crucial in improving both the durability and alignment of the scaffold. However, the thickness of the scaffolds cannot be scaled to the same degree as the other two scaffold dimensions. Future experiments and modifications focusing on more efficiently increasing the thickness of the scaffold, such as changing the gap distance between the spindle and reservoir, or determining how to adhere two scaffolds together, are necessary to further enhance the utility of this material.

Finally, because scaffold formation is dependent on initial solution viscosity, the operating parameters can be adjusted (e.g., shear rate) for the system to account for compositional changes that would enable the addition of other components during fiber formation. FN was utilized as a proof-of-concept molecule, given its ability to unfold under shear and known co-dependent relationship with COL, to demonstrate that other biomolecules can be integrated into the scaffold along with the COL. Rinsing the FN-COL scaffold in sodium deoxycholate indicated that the FN present in the scaffold was indeed fibrillar. Although the exact alignment of these FN fibers is unknown, there is a plethora of evidence from the literature that suggests that the FN fibers are in alignment with the COL fibrils. For example, Ejim et al. used the shear flow generated from a stirred cell to trigger FN fibrillogenesis and create mats of FN. Results showed that the orientation of the FN fibers was in the direction of the flow, much like the COL fibrils in the material. In addition, Paten et al. demonstrated that in a cell-free environment, collagen polymerization-initiated FN fibrillogenesis, and more importantly, that there was a high degree of colocalization between the two protein fibers; since the setup produced highly aligned COL fibrils, it is likely that the FN fibers in the scaffold match the orientation of the COL fibrils. Future studies will be aimed at expanding the repertoire of biomolecules that can be added to these scaffolds while including an assessment of their biological performance in vitro.

### Conclusions

Overall, a scalable platform that leverages the natural polymerization process of COL to reproducibly generate scaffolds with highly aligned fibril substructures has been developed. Parameters in the system, such as shear rate, polymerization time, and concentration were explored and adjusted to both optimize the fibril alignment in the material and understand the process behind the uniaxial fibril orientation. Furthermore, CG molecular dynamics simulations were utilized to understand the initial stage of the alignment process where the COL monomers are first introduced to shear flow. This system is tunable, as it produces structures at multiple dimensions without sacrificing fibril structure and alignment, both of which closely mimic collagenous fibrils from isolated rat tail tendon. Perhaps most interesting is that previous layers of aligned fibrils further improve the alignment of future layers of fibrils up to a degree. Future work consists of developing a more efficient approach to increasing the thickness of the material. Furthermore, while these scaffolds currently consist of layered, type I COL fibrils, preliminary data suggests the immediate potential to incorporate a broad spectrum of biological molecules for tuning the scale and types of tissues formed. These features enable a streamlined pathway to generate customizable materials for future tissue engineering applications.

### Example 2. Design and Production of Customizable and Highly Aligned Fibrillar Collagen Scaffolds

### Abstract

The ability to rapidly and reproducibly fabricate anisotropic collagenous networks has remained elusive despite decades of research. Balancing its propensity to spontaneously polymerize in vitro with the mild processing conditions necessary to maintain its native substructure introduces challenges that are not easily amenable to off-the-shelf instrumentation. To overcome these challenges, a customizable platform that simultaneously builds and aligns type I collagen (COL) fibers as the proteins spontaneously polymerize under mild shear forces has been designed. The mechanism of fibril alignment, targeting variables such as shear rate, viscosity, and time was explored. These variables were further elucidated with coarse-grain simulations, where the threshold values and parameters required to trigger shear-induced alignment that correlated well with experimental findings were calculated. The fibrous materials also showed comparable alignment and mechanics to native rat-tail tendon, illustrating a unique alternative when exploring future tissue engineering applications.

### Introduction

The extracellular matrix (ECM) is a complex, multi-component network that provides the mechanical strength, instructional cues, and underlying structure of tissues and organs in the body. These networks are customized for region-specific architectures, where fiber diameter and organization directly influence tissue form and function (Frantz, C.; Stewart, K. M.; Weaver, V. M., The extracellular matrix at a glance. J Cell Sci 2010, 123 (Pt 24), 4195-200; Hynes, R. O., The extracellular matrix: not just pretty fibrils. Science 2009, 326 (5957), 1216-9; Cowin, S. C., How is a Tissue Built? J Biomech Eng 2000, 122, 553-569). For example, the human Achilles tendon contains uniaxial fibrils made of type I collagen (COL) that resist failure under load, withstanding forces and stresses up to ˜5000 N and ˜80 MP a, respectively (O′Brien, M., Structure and metabolism of tendons. Scand J Med Sci Sports 1997, 7 (2), 55-61; Wren, T. A.; Yerby, S. A.; Beaupre, G. S.; Carter, D. R., Mechanical properties of the human achilles tendon. Clin Biomech (Bristol, Avon) 2001, 16 (3), 245-51). On the other hand, the cornea- also comprised of type I COL fibrils but organized as perpendicular sheets- withstands the intraocular pressure in the eye (-14.2 mmHg) while maintaining optical transparency (Boote, C.; Dennis, S.; Huang, Y.; Quantock, A. J.; Meek, K. M., Lamellar orientation in human cornea in relation to mechanical properties. J Struct Biol 2005, 149 (1), 1-6; Holmes, D. F.; Gilpin, C. J.; Baldock, C.; Ziese, U.; Koster, A. J.; Kadler, K. E., Corneal collagen fibril structure in three dimensions: Structural insights into fibril assembly, mechanical properties, and tissue organization. Proc Natl Acad Sci USA 2001, 98 (13), 7307-12; Hamilton, K. E.; Pye, D. C., Young's modulus in normal corneas and the effect on applanation tonometry. Optom Vis Sci 2008, 85 (6), 445-50; Komai, Y.; Ushiki, T., The three-dimensional organization of collagen fibrils in the human cornea and sclera. Invest Ophthalmol Vis Sci 1991, 32 (8), 2244-58). In an even different manner, the type I COL fibrils in the skin are organized as a woven network that gives this tissue both strength and elasticity (Crichton, M. L.; Chen, X.; Huang, H.; Kendall, M. A., Elastic modulus and viscoelastic properties of full thickness skin characterised at micro scales. Biomaterials 2013, 34 (8), 2087-97). In one study, Yang et al. demonstrated how this basket-weave structure enabled fibrils to straighten, reorient, and slide past one another in order to redistribute external forces and resist tearing under load (Yang, W.; Sherman, V. R.; Gludovatz, B.; Schaible, E.; Stewart, P.; Ritchie, R. O.; Meyers, M. A., On the tear resistance of skin. Nat Commun 2015, 6, 6649). Given the range of unique structure-dependent functions of precisely aligned COL fibrils in vivo, efforts have been made to recapitulate these features in vitro. However, despite decades of research, the ability to control and scale COL I fibrillogenesis in vitro remains a technical challenge. This may be due in part to the fact that the underlying mechanisms regulating COL fibrillogenesis in vivo-specifically the use of auxiliary biomolecules, complex fluid dynamics, and cell-matrix tension as critical contributors of this process- are not well understood (Paten, J. A.; Martin, C. L.; Wanis, J. T.; Siadat, S. M.; Figueroa-Navedo, A. M.; Ruberti, J. W.; Deravi, L. F., Molecular Interactions between Collagen and Fibronectin: A Reciprocal Relationship that Regulates De Novo Fibrillogenesis. Chem 2019, 5 (8), 2126-2145; Martin, C. L.; Bergman, M. R.; Deravi, L. F.; Paten, J. A., A Role for Monosaccharides in Nucleation Inhibition and Transport of Collagen. Bioelectricty 2020, 2 (2), 1-12; Paten, J. A.; Siadat, S. M.; Susilo, M. E.; Ismail, E. N.; Stoner, J. L.; Rothstein, J. P.; Ruberti, J. W., Flow-Induced Crystallization of Collagen: A Potentially Critical Mechanism in Early Tissue Formation. ACS Nano 2016, (5), 5027-40; Holmes, D. F.; Yeung, C. C.; Garva, R.; Zindy, E.; Taylor, S. H.; Lu, Y.; Watson, S.; Kalson, N. S.; Kadler, K. E., Synchronized mechanical oscillations at the cell-matrix interface in the formation of tensile tissue. Proc Natl Acad Sci USA 2018, 115 (40), E9288-E9297).

Despite these challenges, there have been several attempts to generate aligned COL networks in vitro. One common approach involves extruding concentrated COL as fibers or films with the aid of external electric fields (electrospinning), centrifugal forces (rotary jet spinning), differential solvent baths (wet spinning), or 3D-printing (Matthews, J. A.; Wnek, G. E.; Simpson, D. G.; Bowlin, G. L., Electrospinning of collagen nanofibers. Biomacromolecules 2002, 3 (2), 232-8; Zhong, S.; Teo, W. E.; Zhu, X.; Beuerman, R. W.; Ramakrishna, S.; Yung, L. Y., An aligned nanofibrous collagen scaffold by electrospinning and its effects on in vitro fibroblast culture. J Biomed Mater Res A 2006, 79 (3), 456-63; Chantre, C. O.; Gonzalez, G. M.; Ahn, S.; Cera, L.; Campbell, P. H.; Hoerstrup, S. P.; Parker, K. K., Porous Biomimetic Hyaluronic Acid and Extracellular Matrix Protein Nanofiber Scaffolds for Accelerated Cutaneous Tissue Repair. ACS Appl Mater Interfaces 2019, 11 (49), 45498-45510; Caves, J. M.; Kumar, V. A.; Wen, J.; Cui, W.; Martinez, A.; Apkarian, R.; Coats, J. E.; Berland, K.; Chaikof, E. L., Fibrillogenesis in continuously spun synthetic collagen fiber. J Biomed Mater Res B Appl Biomater 2010, 93 (1), 24-38; Yaari, A.; Schilt, Y.; Tamburu, C.; Raviv, U.; Shoseyov, O., Wet Spinning and Drawing of Human Recombinant Collagen. ACS Biomater Sci Eng 2016, 2, 349-360; Lee, A.; Hudson, A. R.; Shiwarski, D. J.; Tashman, J. W.; Hinton, T. J.; Yerneni, S.; Bliley, J. M.; Campbell, P. G.; Feinberg, A. W., 3D bioprinting of collagen to rebuild components of the human heart. Science 2019, 365 (6452), 482-487; Betsch, M.; Cristian, C.; Lin, Y. Y.; Blaeser, A.; Schoneberg, J.; Vogt, M.; Buhl, E. M.; Fischer, H.; Duarte Campos, D. F., Incorporating 4D into Bioprinting: Real-Time Magnetically Directed Collagen Fiber Alignment for Generating Complex Multilayered Tissues. Adv Healthc Mater 2018, 7 (21), e1800894). These extrusion-based methods have demonstrated some potential for scalability in materials applications. For instance, Lee et al. illustrated scalability in 3D-printing a 55×37 mm replicate of a neonatal human heart comprised solely of COL. Despite this incredible feat, it is unclear whether the fibrillar ultrastructure of COL was retained throughout this process. When ultrastructure has been emphasized, it has often been done so at the cost of scalability and application. For instance, Lai et al. utilized shear flow to align acidic COL monomers that then form a fibrillar COL tube when neutralized. In a separate study, Saeidi et al. demonstrated that shear flow can similarly align COL fibrils during the polymerization process in the direction of the flow to form fibrillar networks. Other techniques including microfluidics, flow-induced crystallization, differential electrochemistry, magnetic orientation, spin coating, and nanolithography have seen moderate successes in generating and aligning COL fibrils (Lai, E. S.; Anderson, C. M.; Fuller, G. G., Designing a tubular matrix of oriented collagen fibrils for tissue engineering. Acta Biomater 2011, 7 (6), 2448-56; Saeidi, N.; Sander, E. A.; Ruberti, J. W., Dynamic shear-influenced collagen self-assembly. Biomaterials 2009, 30 (34), 6581-92; Lanfer, B.; Freudenberg, U.; Zimmermann, R.; Stamov, D.; Korber, V.; Werner, C., Aligned fibrillar collagen matrices obtained by shear flow deposition. Biomaterials 2008, 29 (28), 3888-95; Cheng, X.; Gurkan, U. A.; Dehen, C. J.; Tate, M. P.; Hillhouse, H. W.; Simpson, G. J.; Akkus, O., An electrochemical fabrication process for the assembly of anisotropically oriented collagen bundles. Biomaterials 2008, 29 (22), 3278-88; Torbet, J.; Ronziere, M. C., Magnetic alignment of collagen during self-assembly. Biochem J1984, 219 (3), 1057-9; Saeidi, N.; Sander, E. A.; Zareian, R.; Ruberti, J. W., Production of highly aligned collagen lamellae by combining shear force and thin film confinement. Acta Biomater 2011, 7 (6), 2437-47; Wilson, D. L.; Martin, R.; Hong, S.; Cronin-Golomb, M.; Mirkin, C. A.; Kaplan, D. L., Surface organization and nanopatterning of collagen by dip-pen nanolithography. Proc Natl Acad Sci USA 2001, 98 (24), 13660-4). However, the majority of these procedures lack the scalability required to develop full tissue replicates.

To improve scalability without sacrificing fibrillar ultrastructure, many researchers have focused on re-orienting and aligning pre-assembled fibrils with the aid of magnetic bead-induced alignment, biaxial gel compression, or counter-rotating extrusion (Antman-Passig, M.; Shefi, O., Remote Magnetic Orientation of 3D Collagen Hydrogels for Directed Neuronal Regeneration. Nano Lett 2016, 16 (4), 2567-73; Guo, C.; Kaufman, L. J., Flow and magnetic field induced collagen alignment. Biomaterials 2007, 28 (6), 1105-14; Zitnay, J. L.; Reese, S. P.; Tran, G.; Farhang, N.; Bowles, R. D.; Weiss, J. A., Fabrication of dense anisotropic collagen scaffolds using biaxial compression. Acta Biomater 2018, 65, 76-87; Hoogenkamp, H. R.; Bakker, G. J.; Wolf, L.; Suurs, P.; Dunnewind, B.; Barbut, S.; Friedl, P.; van Kuppevelt, T. H.; Daamen, W. F., Directing collagen fibers using counter-rotating cone extrusion. Acta Biomater 2015, 12, 113-121; Yang, S.; Shi, X.; Li, X.; Wang, J.; Wang, Y.; Luo, Y., Oriented collagen fiber membranes formed through counter-rotating extrusion and their application in tendon regeneration. Biomaterials 2019, 207, 61-75). Perhaps the most readily scalable of the three is counter-rotating extrusion, which was originally designed and utilized to prepare synthetic sausage casings but has since been adapted for tissue engineering applications (Yang, S.; Wang, J.; Wang, Y.; Luo, Y., Key role of collagen fibers orientation in casing-meat adhesion. Food Res Int 2016, 89 (Pt 1), 439-447; Harper, B. A.; Barbut, S.; Lim, L.-T.; Marcone, M. F., Microstructural and textural investigation of various manufactured collagen sausage casings. Food Res 2012, 49, 494-500). This method consists of concentric cones rotating in opposite directions, effectively molding a preformed COL dough into aligned sheets, where the shear from the cones is believed to pull COL fibrils in one direction to induce alignment. Yang et al. and Hoogenkamp et al. describe aligned networks that persist over centimeter-scale lengths which are at least ten times greater than the other previously mentioned strategies. Lastly, the authors describe the utility of these molded scaffolds in a rat Achilles tendon surgery, illustrating their potential in promoting tenogenic differentiation for tendon repair.

Despite the successes associated with counter-rotating extrusion, the long-term impact of such brut tissue molding and fibril reconstruction on structure stability and function remains unknown. The question is whether it would be feasible to reconfigure this approach in order to eliminate the need for a preformed COL dough as a starting material. To test this, a device that consists of two concentric cylinders: an outer stationary cylinder and a rotating inner cylinder was designed. This system employs Couette flow to a solution of COL monomers that simultaneously polymerize and align under shear fluid flow to create continuous fiber networks. The effect of shear stress, shear rate, and concentration on COL fibril anisotropy and describe underlying mechanisms potentiating this alignment was explored, as well as the stability of the COL networks and their mechanics. Finally, given the ease of the system, the scalability of the method, illustrating a facile approach to producing COL fibers in vitro, was demonstrated.

### Methods

Unless otherwise specified, the materials and methods are the same or substantially the same as those used in Example 1.

### Results and Discussion

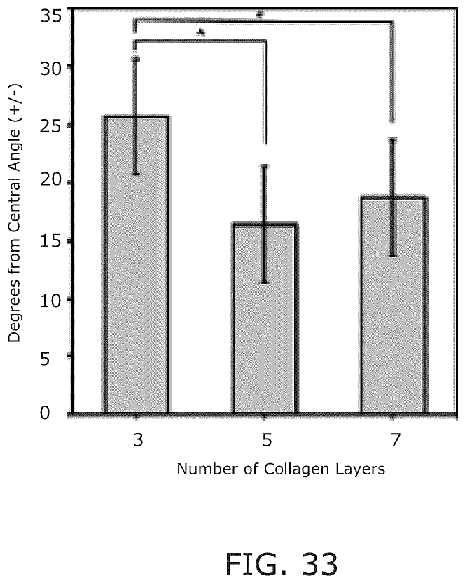

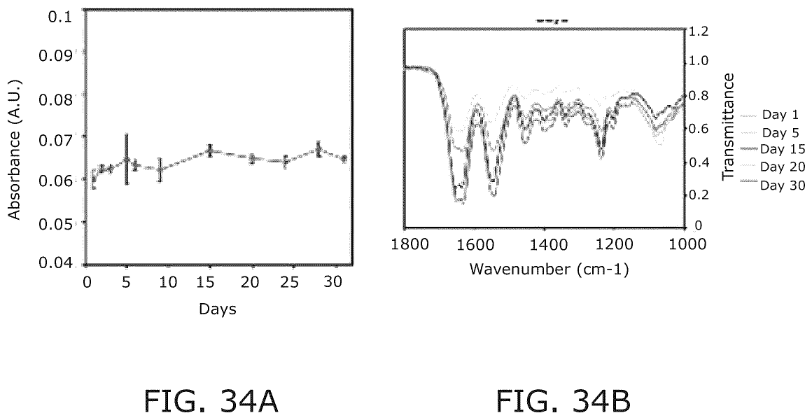

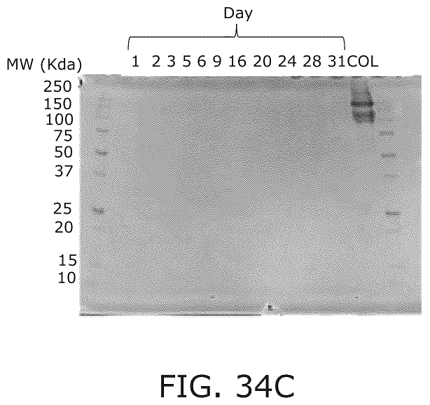

To generate sheets of COL fibrils, a device with two concentric cylinders, where the inner cylinder (the spindle) rotated, and the outer cylinder (the reservoir) remained stationary (FIG. 1) was designed. This setup allowed for neutralized COL to spontaneously polymerize under shear flow, leading to the formation of the fibrillar network. Prior to spinning, the spindle was coated in a thin, uniform layer of gelatin (FIGS. 21A-C). Without the gelatin layer, polymerized COL formed only as a thin layer at the air-liquid interface (FIG. 21A). In the presence of the gelatin, COL fibrils spanned the entire spindle length, enabling their collection as a ˜3×1 cm scaffold (FIG. 2B and FIG. 21B). It is likely that the functional groups present on gelatin undergo strong intermolecular interactions (such as hydrogen bonding) with those on the COL, promoting adhesion during the spinning process; without these functional groups, COL is unable to bind and polymerize on the spindle. Perhaps more importantly, the gelatin served as a sacrificial layer that allowed for an easy removal of the scaffold from the spindle without disrupting the anisotropic substructure (FIGS. 2B-C). Indeed, the exquisite alignment that persisted throughout the scaffolds was comparable to those found in rat tail tendons (FIGS. 33A-C and FIGS. 34A-B), and the presence of the D-banding suggests that the shear-induced alignment did not disrupt the natural polymerization of COL (FIG. 2C).

To examine the impact of tethering on the dynamic alignment of COL molecules under shear, a CG simulation framework was constructed to qualitatively model and simulate molecules with one bead tethered at one end of the chain. In this configuration, simulations emulated the initial deformation of the COL molecules, which was defined as the uncoiling and alignment of the molecules on the spindle. In these conditions, it was observed that a single COL molecule gradually aligns within approximately 3 ns when the magnitude of the tethering force was above a critical threshold of 0.005 kcal/mol/Å (FIGS. 24A-D). When the tethering force was below this threshold, the tethering force did not overcome the pairwise, long-range cohesive/attractive forces between two interacting beads, thus resulting in an unaligned structure. Above the threshold, the tethering force would easily surmount these opposing forces and possibly initiate the uncoiling of the COL molecule, which eventually led to the aligned structure.

To expand on these findings, the aligning threshold force for single COL chains that were 5- and 10-molecules long and observed an increase in threshold force as the length of the COL chains grew (FIGS. 5A-C) was calculated. This increase was required to overcome the larger intramolecular adhesion and pairwise attractive forces that intensified due to the increased numbers of CG beads. Interestingly, it was also found that the critical force needed to align multiple chains of COL was the same as that of a single molecule-long COL (FIGS. 6A-C and FIGS. 7A-C) This was in contrast to the larger critical force needed to align a single chain of a much longer collagen molecule. This difference was attributed to the intramolecular stiffness and entanglement that corresponded with the increased length of a chain that ultimately prevented the COL backbone from moving freely. In contrast, intermolecular interactions, such as shearing between multiple chains of shorter COL molecules, appeared to be beneficial for overcoming the backbone stiffness and entanglement, which led to a competition between intra- and intermolecular effects. These simulation results were also consistent with experimental findings, where it was observed that alignment is lost, if the initial solutions are not sheared immediately (FIGS. 8A-C; FIGS. 9A-C; and FIGS. 10A-B). When taken together, the computational and experimental findings suggest that a combination of larger critical forces and longer spinning times would be necessary to align the pre-polymerized COL networks.

In agreement with the hypothesis that intermolecular interactions compete against the individual molecules' tendency towards misalignment, it was also found that overall alignment of the system can be achieved without the need to tether every available chain (FIGS. 7A-C). In a 625-chain system, overall alignment was achieved with both 100% (FIG. 6C) and 66% chains tethered, whereas misalignment reigned with only 33% tethered. This difference indicated that a minimal number of tethered chains were required to overcome entropic effects. Therefore, starting solutions consisting of concentrated (e.g., more available chains) monomers (e.g., shorter COL molecules) should align with greater ease aided by the denser space and assistive shear forces from surrounding tethered and aligned molecules. Once alignment starts, it may also facilitate the uncoiling of neighboring unaligned molecules via intermolecular repulsion when they are close to each other as a result of limited wiggle room. This hypothesis was supported by the steep increase in P₂ in FIGS. 7A-C, which denotes the structural evolution of the tethered molecules, followed by the gradual increase in P₂ which was primarily due to the structural changes in the untethered molecules that are driven to align together with the tethered chains. Hence, at the initiation of spinning in bulk COL solutions, only a few molecules needed to be tethered and aligned to drive the overall system-wide alignment. From the molecular perspective, these results highlighted the importance of the gelatin layer (i.e., the tether) and simultaneous spinning/polymerization of COL to achieve eventual alignment.

Processing conditions may influence the physical features of the scaffold. To test this, the ideal number of layers required for the scaffolds to remain intact was investigated. To test this, features such as scaffold thickness, fibril alignment, and porosity of scaffolds composed of 3-,5-, and 7-layer scaffolds (FIGS. 15, 22, 29, 35) were explored. Interestingly, the addition of more layers of COL only minimally increased the thickness of the scaffold (369, 625, and 813 nm for 3-, 5-, and 7- layers of COL respectively (FIG. 15)). Furthermore, when the shear rate was held constant, fibril alignment increased when the number of COL layers increased from 3 to 5 (FIG. 29), suggesting that previous layers partially serve as a template for new fibrils. However, there was no significant improvement in fibril alignment or scaffold porosity between the 5- and 7- layer scaffolds (FIG. 22). Given the overall negligible differences between 5- and 7-layer scaffolds, with the only a minimal increase in scaffold thickness, all remaining experiments were conducted with the 5-layer scaffolds.

In order to evaluate how the shear rate of the solution impacted COL fibril alignment, shear rates of 50, 75, 100 and 1000 s⁻¹ were tested. At these rates, it was calculated that the fibril angle variances were ±20.7°, ±16.3°, ±17.0°, and ±25.7° respectively (FIGS. 26A-D and FIGS. 27A-C). At shear rates above 1000 s⁻¹, the gelatin was torn off the spindle, preventing network formation. Without shear, the network was completely isotropic (FIGS. 33A-C and FIGS. 34A-B). These results suggest that application of shear, at any rate below 1000 s⁻¹ was sufficient to induce broad area alignment across the scaffold (FIGS. 28A-B). However, fibril organization was greatest at 75 s⁻¹, and the most similar to the alignment measurements of COL scaffolds from other published work. Statistical analysis also revealed that there is no significant difference between the fibril alignment in any of the scaffolds prepared at different shear rates, and the fibril alignment in the rat tail tendon (FIGS. 33A-C and FIGS. 34A-B). Furthermore, the low Reynold's numbers at these shear rates suggest that the flow the rotating spindle creates is laminar, which likely contributed to the production of aligned fibrils (Table 2).

TABLE 2
Reynold's Number in Concentric Cylinder Device

| Shear Rate (s⁻¹) | Reynold's Number |
|---|---|
| 50 | 1.5 |
| 75 | 2.3 |
| 100 | 3.0 |
| 1000 | 21.9 |

Because negligible changes to fibril alignment as a function of shear rate were observed, how shear stress influenced alignment at multiple concentrations of COL was tested. Changing the concentration of the initial COL solution alters the viscosity of the solution. Because shear stress is directly related to the shear rate and viscosity, it was approximated that to maintain a constant shear stress of 1.7 N/m² (the amount of shear the system applies at 75 s⁻¹), the rotational speed for solutions prepared with 0.5, 1.5, and 2.5 mg/mL COL (FIG. 11) must be adjusted. It was observed that by maintaining constant shear stress, statistically similar alignment for each condition was able to be achieved (±23.0°, ±24.7°, and ±16.3° for 0.5, 1.5, and 2.5 mg/mL, respectively FIGS. 12A-C). These findings supported that shear stress is a critical component in fibril alignment and even though other parameters were altered (i.e., shear rate and concentration), alignment and fibril diameter were maintained throughout the scaffolds.

Given the tunability of the scaffolds and their structural similarity to the commonly tested rat-tail tendons (RTTs) (FIGS. 33A-C and FIGS. 34A-B), the maximum force and failure strain of both hydrated and dried scaffolds compared to hydrated and dried RTTs was tested. The hydrated scaffolds only withstood an average force of 0.023±0.005 N but experienced a strain of 20.3±8.4% before breakage (FIGS. 31A-B and FIGS. 32A-B). Furthermore, the scaffold tore apart slowly, and after the initial break, the scaffold was still able to withstand partial load before complete failure. On the other hand, the dried scaffolds experienced an average load of 1.18 35 0.23 N before breakage, a two-order of magnitude increase compared to the hydrated scaffolds, albeit at a significantly lower strain of 9.96±3.84%.

The mechanical characteristics of these scaffolds were compared to a hydrated and dried tendon extracted from a rat tail. The hydrated tendon performed similarly to the dried scaffold, with an average maximum load of 1.34±0.15 N and average maximum strain of 9.40±1.80%, but the dried scaffold was able to withstand both a greater average force of 3.20±0.27 N before breakage and strain of 23.0±1.92%. It is likely that the hydrated scaffolds were only able to sustain a fraction of the force the tendons withstood due to a lack of natural cross-linking. It is possibly that in the scaffolds the fibrils were sliding by each other when placed under a load, which would account for a lower maximum force but a higher strain. The fibrils in the dehydrated scaffold possibly adhered together when dried and became more of a dense material, which may be responsible for the scaffold being able to experience a greater force.

Because of the ease of manufacturing, it was tested how scalable and versatile this platform is. To test this, the dimensions of the device were increased by 50% which, as a result, increased the size of the scaffold to approximately 4.5×1.5 cm (FIG. 30C). This increase in size did not significantly alter the fiber alignment throughout the scaffold. On average, the fibrillar alignment for the large scaffold was ±14.6°, compared to the ±16.3° alignment on the smaller scaffold, indicating that scaling this design was feasible without sacrificing fiber structure or alignment.

Finally, it was tested whether the shear-induced polymerization was amenable to other fibrillar proteins. To test this, soluble FN was blended into the starting COL solution and formed scaffolds. It was observed that the presence of fibril FN that persisted throughout the scaffolds (FIGS. 30A-B). To confirm that the FN was indeed in its fibrillar form, the scaffold was incubated in 2% sodium deoxycholate, a chemical that digests globular FN but not fibrillar FN, and observed FN positive fluorescence, indicating that fibrillar FN was present in the scaffold (FIG. 30D).

### Conclusions

A platform that uses a rotating concentric cylinder to establish a unique set of fluid dynamics, termed Couette-flow, to drive the rapid alignment and polymerization of protein networks was developed. Parameters in the system such as shear rate, time, and protein concentration were adjusted to determine the conditions that lead networks with the greatest alignment throughout the entire material. The mechanical properties of the network were explored and highlighted that the method was scalable. Lastly, another extracellular matrix protein was integrated into the network to produce multi-protein scaffolds and aid in biocompatibility. Overall, this system provides an efficient, customizable, and highly reproducible platform to build a network of aligned COL fibrils that not only mimics the anisotropy of native rat tail tendon but also approaches its mechanical properties. While the scaffolds currently consist of layered, type I COL fibrils, preliminary data suggests the immediate potential to incorporate a broad spectrum of biological molecules for scalable tissue formation.

### REFERENCES

1. Frantz, C.; Stewart, K. M.; Weaver, V. M., The extracellular matrix at a glance. J. Cell. Sci. 2010, 123 (Pt 24), 4195-200. DOI: 10.1242/jcs.023820.
2. Hynes, R. OScience 2009, 326 (5957), 1216-9. DOI: 10.1126/science.1176009.
3. Cowin, S. C., How is a Tissue Built? J. Biomech. Eng. 2000, 122, 553-569.
4. O'Brien, M., Structure and metabolism of tendons. Scand. J. Med. Sci. Sports 1997, 7 (2), 55-61. DOI: 10.1111/j.1600-0838.1997.tb00119.x.
5. Wren, T. A.; Yerby, S. A.; Beaupre, G. S.; Carter, D. R., Mechanical properties of the human achilles tendon. Clin. Biomech. (Bristol, Avon) 2001, 16 (3), 245-51. DOI: 10.1016/s0268-0033(00)00089-9.
6. Boote, C.; Dennis, S.; Huang, Y; Quantock, A. J.; Meek, K. M., Lamellar orientation in human cornea in relation to mechanical properties. J. Struct. Biol. 2005, /49 (1), 1-6. DOI: 10.1016/j.jsb.2004.08.009.
7. Holmes, D. F.; Gilpin, C. J.; Baldock, C.; Ziese, U.; Koster, A. J.; Kadler, K. E., Corneal collagen fibril structure in three dimensions: Structural insights into fibril assembly, mechanical properties, and tissue organization. Proc. Natl. Acad. Sci. U.S.A. 2001, 98 (13), 7307-12. DOI: 10.1073/pnas.111150598.
8. Hamilton, K. E.; Pye, D. C., Young's modulus in normal corneas and the effect on applanation tonometry. Optom. Vis. Sci. 2008, 85 (6), 445-50. DOI: 10.1097/OPX.0b013e3181783a70.
9. Komai, Y; Ushiki, T., The three-dimensional organization of collagen fibrils in the human cornea and sclera. Invest. Ophthalmol. Vis. Sci. 1991, 32 (8), 2244-58.
10. Crichton, M. L.; Chen, X.; Huang, H.; Kendall, M. A., Elastic modulus and viscoelastic properties of full thickness skin characterised at micro scales. Biomaterials 2013, 34 (8), 2087-97. DOI: 10.1016/j .biomaterials.2012.11.035.
11. Yang, W.; Sherman, V. R.; Gludovatz, B.; Schaible, E.; Stewart, P.; Ritchie, R. 0.; Meyers, M. A., On the tear resistance of skin. Nat. Commun. 2015, 6, 6649. DOI: 10.1038/ncomms7649.
12. Kleinman, H. K.; Martin, G. R., Matrigel: Basement membrane matrix with biological activity. Semin. Cancer Biol. 2005, 15 (5), 378-386. DOI: 10.1016/j.semcancer.2005.05.004.
13. Nerger, B. A.; Brun, P.-T.; Nelson, C. M., Microextrusion printing cell-laden networks of type I collagen with patterned fiber alignment and geometry. Soft Matter 2019, 15 (5728).
14. Ghasemi-Mobarakeh, L.; Prabhakaran, M. P.; Morshed, M.; Nasr-Esfahani, M. H.; Ramakrishna, S., Bio-functionalized PCL nanofibrous scaffolds for nerve tissue engineering. Mater. Sci. Eng. C-Mater. 2010, 30 (8), 1129-1136. DOI: 10.1016/j.msec.2010.06.004.
15. Ejim, O. S.; Blunn, G. W.; Brown, R. A., Production of artifical-oriented mats and strands from plasma fibronectin: a morphological study. Biomaterials 1993, 14.
16. Chantre, C. O.; Campbell, P. H.; Golecki, H. M.; Buganza, A. T.; Capulli, A. K.; Deravi, L. F.; Dauth, S.; Sheehy, S. P.; Paten, J. A.; Gledhill, K.; Doucet, Y. S.; Abaci, H. E.; Ahn, S.; Pope, B. D.; Ruberti, J. W.; Hoerstrup, S. P.; Christiano, A. M.; Parker, K. K., Production-scale fibronectin nanofibers promote wound closure and tissue repair in a dermal mouse model. Biomaterials 2018, 166, 96-108. DOI: 10.1016/j.biomaterial s.2018.03.006.
17. Chantre, C. O.; Gonzalez, G. M.; Ahn, S.; Cera, L.; Campbell, P. H.; Hoerstrup, S. P.; Parker, K. K., Porous Biomimetic Hyaluronic Acid and Extracellular Matrix Protein Nanofiber Scaffolds for Accelerated Cutaneous Tissue Repair. ACS Appl. Mater. Interfaces 2019, 11 (49), 45498-45510. DOI: 10.1021/acsami.9b17322.
18. Buttafoco, L.; Kolkman, N. G.; Engbers-Buijtenhuijs, P.; Poot, A. A.; Dijkstra, P. J.; Vermes, I.; Feijen, J., Electrospinning of collagen and elastin for tissue engineering applications. Biomaterials 2006, 27 (5), 724-734. DOI: 10.1016/j.biomaterials.2005.06.024.
19. Paten, J. A.; Martin, C. L.; Wanis, J. T.; Siadat, S. M.; Figueroa-Navedo, A. M.; Ruberti, J. W.; Deravi, L. F., Molecular Interactions between Collagen and Fibronectin: A Reciprocal Relationship that Regulates De Novo Fibrillogenesis. Chem 2019, 5 (8), 2126-2145. DOI: https://doi.org/10.1016/j.chempr.2019.05.011.
20. Martin, C. L.; Bergman, M. R.; Deravi, L. F.; Paten, J. A., A Role for Monosaccharides in Nucleation Inhibition and Transport of Collagen. Bioelectricty 2020, 2 (2), 1-12.
21. Paten, J. A.; Siadat, S. M.; Susilo, M. E.; Ismail, E. N.; Stoner, J. L.; Rothstein, J. P.; Ruberti, J. W., Flow-Induced Crystallization of Collagen: A Potentially Critical Mechanism in Early Tissue Formation. ACS Nano 2016, 10 (5), 5027-40. DOI: 10.1021/acsnano.5b07756.
22. Holmes, D. F.; Yeung, C. C.; Garva, R.; Zindy, E.; Taylor, S. H.; Lu, Y.; Watson, S.; Kalson, N. S.; Kadler, K. E., Synchronized mechanical oscillations at the cell-matrix interface in the formation of tensile tissue. Proc. Natl. Acad. Sci. U.S.A. 2018, 115 (40), E9288-E9297. DOI: 10.1073/pnas.1801759115.
23. Matthews, J. A.; Wnek, G. E.; Simpson, D. G.; Bowlin, G. L., Electrospinning of collagen nanofibers. Biomacromolecules 2002, 3 (2), 232-8. DOI: 10.1021/bm015533u.
24. Zhong, S.; Teo, W. E.; Zhu, X.; Beuerman, R. W.; Ramakrishna, S.; Yung, L. Y., An aligned nanofibrous collagen scaffold by electrospinning and its effects on in vitro fibroblast culture. J. Biomed. Mater. Res. A 2006, 79 (3), 456-63. DOI: 10.1002/jbm.a.30870.
25. Caves, J. M.; Kumar, V. A.; Wen, J.; Cui, W.; Martinez, A.; Apkarian, R.; Coats, J. E.; Berland, K.; Chaikof, E. L., Fibrillogenesis in continuously spun synthetic collagen fiber. J. Biomed. Mater. Res. B Appl. Biomater 2010, 93 (1), 24-38. DOI: 10.1002/jbm.b.31555.
26. Yaari, A.; Schilt, Y; Tamburu, C.; Raviv, U.; Shoseyov, O., Wet Spinning and Drawing of Human Recombinant Collagen. ACS Biomater Sci. Eng. 2016, 2, 349-360.
27. Lee, A.; Hudson, A. R.; Shiwarski, D. J.; Tashman, J. W.; Hinton, T. J.; Yerneni, S.; Bliley, J. M.; Campbell, P. G.; Feinberg, A. W., 3D bioprinting of collagen to rebuild components of the human heart. Science 2019, 365 (6452), 482-487. DOI: 10.1126/science.aav9051.
28. Betsch, M.; Cristian, C.; Lin, Y. Y.; Blaeser, A.; Schoneberg, J.; Vogt, M.; Buhl, E. M.; Fischer, H.; Duarte Campos, D. F., Incorporating 4D into Bioprinting: Real-Time Magnetically Directed Collagen Fiber Alignment for Generating Complex Multilayered Tissues. Adv. Healthc. Mater. 2018, 7 (21), e1800894. DOI: 10.1002/adhm.201800894.
29. Zeugolis, D. I.; Khew, S. T.; Yew, E. S.; Ekaputra, A. K.; Tong, Y W.; Yung, L. Y.; Hutmacher, D. W.; Sheppard, C.; Raghunath, M., Electro-spinning of pure collagen nano-fibres—just an expensive way to make gelatin? Biomaterials 2008, 29 (15), 2293-305. DOI: 10.1016/j .biomaterials.2008.02.009.
30. Rogalski, J. J.; Bastiaansen, C. W. M.; Peijs, T., Rotary jet spinning review- a potential high yield future for polymer nanofibers. Nanocomposites 2017, 3 (4), 97-121.
31. Bhardwaj, N.; Kundu, S. C., Electrospinning: a fascinating fiber fabrication technique. Biotechnol. Adv. 2010, 28 (3), 325-47. DOI: 10.1016/j.biotechadv.2010.01.004.
32. Saeidi, N.; Sander, E. A.; Ruberti, J. W., Dynamic shear-influenced collagen self-assembly. Biomaterials 2009, 30 (34), 6581-92. DOI: 10.1016/j .biomaterials.2009.07.070.
33. Lanfer, B.; Freudenberg, U.; Zimmermann, R.; Stamov, D.; Korber, V; Werner, C., Aligned fibrillar collagen matrices obtained by shear flow deposition. Biomaterials 2008, 29 (28), 3888-95. DOI: 10.1016/j .biomaterials.2008.06.016.
34. Cheng, X.; Gurkan, U. A.; Dehen, C. J.; Tate, M. P.; Hillhouse, H. W.; Simpson, G. J.; Akkus, O., An electrochemical fabrication process for the assembly of anisotropically oriented collagen bundles. Biomaterials 2008, 29 (22), 3278-88. DOI: 10.1016/j.biomaterials.2008.04.028.
35. Torbet, J.; Ronziere, M. C., Magnetic alignment of collagen during self-assembly. Biochem. 1 1984, 219 (3), 1057-9. DOI: 10.1042/bj2191057.
36. Saeidi, N.; Sander, E. A.; Zareian, R.; Ruberti, J. W., Production of highly aligned collagen lamellae by combining shear force and thin film confinement. Acta Biomater 2011, 7 (6), 2437-47. DOI: 10.1016/j .actbio.2011.02.038.
37. Wilson, D. L.; Martin, R.; Hong, S.; Cronin-Golomb, M.; Mirkin, C. A.; Kaplan, D. L., Surface organization and nanopatterning of collagen by dip-pen nanolithography. Proc. Natl. Acad. Sci. U.S.A. 2001, 98 (24), 13660-4. DOI: 10.1073/pnas.241323198.
38. Antman-Passig, M.; Shefi, O., Remote Magnetic Orientation of 3D Collagen Hydrogels for Directed Neuronal Regeneration. Nano Lett. 2016, 16 (4), 2567-73. DOI: 10.1021/acs.nanolett.6b00131.
39. Guo, C.; Kaufman, L. J., Flow and magnetic field induced collagen alignment. Biomaterials 2007, 28 (6), 1105-14. DOI: 10.1016/j .biomaterials.2006.10.010.
40. Zitnay, J. L.; Reese, S. P.; Tran, G.; Farhang, N.; Bowles, R. D.; Weiss, J. A., Fabrication of dense anisotropic collagen scaffolds using biaxial compression. Acta Biomater 2018, 65, 76-87. DOI: 10.1016/j.actbio.2017.11.017.
41. Hoogenkamp, H. R.; Bakker, G. J.; Wolf, L.; Suurs, P.; Dunnewind, B.; Barbut, S.; Friedl, P.; van Kuppevelt, T. H.; Daamen, W. F., Directing collagen fibers using counter-rotating cone extrusion. Acta Biomater 2015, 12, 113-121. DOI: 10.1016/j.actbio.2014.10.012.
42. Yang, S.; Shi, X.; Li, X.; Wang, J.; Wang, Y; Luo, Y, Oriented collagen fiber membranes formed through counter-rotating extrusion and their application in tendon regeneration. Biomaterials 2019, 207, 61-75. DOI: 10.1016/j.biomaterials.2019.03.041.
43. Yang, S.; Wang, J.; Wang, Y.; Luo, Y, Key role of collagen fibers orientation in casing-meat adhesion. Food Res. Int. 2016, 89 (Pt 1), 439-447. DOI: 10.1016/j.foodres.2016.08.035.
44. Harper, B. A.; Barbut, S.; Lim, L.-T.; Marcone, M. F., Microstructural and textural investigation of various manufactured collagen sausage casings. Food Res. 2012, 49, 494-500.
45. Depalle, B.; Qin, Z.; Shefelbine, S. J.; Buehler, M. J., Influence of cross-link structure, density and mechanical properties in the mesoscale deformation mechanisms of collagen fibrils. J. Mech. Behay. Biomed. Mater. 2015, 52, 1-13.
46. Buehler, M. J., Atomistic and continuum modeling of mechanical properties of collagen: elasticity, fracture, and self-assembly. J. Mater. Res. 2006, 21 (8), 1947-1961.
47. Gautieri, A.; Russo, A.; Vesentini, S.; Redaelli, A.; Buehler, M. J., Coarse-grained model of collagen molecules using an extended MARTINI force field. J. Chem. Theory Comput. 2010, 6 (4), 1210-1218.
48. Zhang, N.; Cheng, Y; Hu, X.; Yeo, J., Toward rational algorithmic design of collagen-based biomaterials through multiscale computational modeling. Curr. Opin. Chem. Eng. 2019, 24, 79-87.
49. Buehler, M. J.; Wong, S. Y, Entropic elasticity controls nanomechanics of single tropocollagen molecules. Biophy. 1 2007, 93 (1), 37-43.
50. Buehler, M. J., Nanomechanics of collagen fibrils under varying cross-link densities: atomistic and continuum studies. J. Mech. Behay. Biomed. Mater. 2008, 1 (1), 59-67.
51. Buehler, M. J., Nature designs tough collagen: explaining the nanostructure of collagen fibrils. Proc. Natl. Acad. Sci. U.S.A. 2006, 103 (33), 12285-12290.
52. Gautieri, A.; Buehler, M. J.; Redaelli, A., Deformation rate controls elasticity and unfolding pathway of single tropocollagen molecules. J. Mech. Behay. Biomed. Mater. 2009, 2 (2), 130-137.
53. Depalle, B.; Qin, Z.; Shefelbine, S. J.; Buehler, M. J., Large deformation mechanisms, plasticity, and failure of an individual collagen fibril with different mineral content. J. Bone and Miner. Res. 2016, 31 (2), 380-390.
54. Sander, E. A.; Barocas, V. H., Comparison of 2D fiber network orientation measurement methods. J. Biomed. Mater. Res. A 2009, 88 (2), 322-31. DOI: 10.1002/jbm.a.31847.
55. Orgel, J. P.; Irving, T. C.; Miller, A.; Wess, T. J., Microfibrillar structure of type I collagen in situ. Proc. Natl. Acad. Sci. U.S.A. 2006, 103 (24), 9001-9005.
56. Shoulders, M. D.; Raines, R. T., Collagen structure and stability. Annu. Rev. Biochem. 2009, 78, 929-58. DOI: 10.1146/annurev.biochem.77.032207.120833.
57. Yong, C.; Higgs, P., Chain orientation in polymer networks: Computer simulations using the bond fluctuation model. Macromolecules 1999, 32 (15), 5062-5071.
58. Liu, J.; Yang, R., Tuning the thermal conductivity of polymers with mechanical strains. Phys. Rev. B 2010, 81 (17), 174122.
59. Allen, M. P.; Tildesley, D. J., Computer simulation of liquids. Oxford university press: 2017.
60. Lai, E. S.; Anderson, C. M.; Fuller, G. G., Designing a tubular matrix of oriented collagen fibrils for tissue engineering. Acta Biomater 2011, 7 (6), 2448-56. DOI: 10.1016/j.acbio.2011.03.012.
61. de Wild, M.; Pomp, W.; Koenderink, G. H., Thermal memory in self-assembled collagen fibril networks. Biophys. 1 2013, 105 (1), 200-10. DOI: 10.1016/j.bpj.2013.05.035.
62. Liu, G. Y.; Agarwal, R.; Ko, K. R.; Ruthven, M.; Sarhan, H. T.; Frampton, J. P., Templated Assembly of Collagen Fibers Directs Cell Growth in 2D and 3D. Sci. Rep. 2017, 7.
63. Kapoor, A.; Caporali, E. H. G.; Kenis, P. J. A.; Stewart, M. C., Microtopographically patterned surfaces promote the alignment of tenocytes and extracellular collagen. Acta Biomater 2010, 6, 2580-2589.
64. Kubow, K. E.; Vukmirovic, R.; Zhe, L.; Klotzsch, E.; Smith, M. L.; Gourdon, D.; Luna, S.; Vogel, V, Mechanical forces regulate the interactions of fibronectin and collagen I in extracellular matrix. Nat. Commun. 2015, 6.
65. Li, S.; Van Den Diepstraten, C.; D'Souza, S. J.; Chan, B. M.; Pickering, J. G., Vascular smooth muscle cells orchestrate the assembly of type I collagen via alpha2beta1 integrin, RhoA, and fibronectin polymerization. Am. J. Pathol. 2003, 163 (3), 1045-56. DOI: 10.1016/s0002-9440(10)63464-5.

The foregoing Example has been described in Martin, Cassandra & Zhai, Chenxi & Paten, Jeffrey & Yeo, Jingjie & Deravi, Leila. (2021). Design and Production of Customizable and Highly Aligned Fibrillar Collagen Scaffolds. ACS Biomaterials Science & Engineering. 10.1021/acsbiomaterials.1c00566. The contents of this reference and its supplemental information and materials are incorporated herein by reference in their entirety.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed:

1. A fibrillar polymeric scaffold comprising fibrils derived from one or more polymerizable monomers, wherein the scaffold is non-cellular and about 50% or greater of the fibrils are within ±20° of the orientation of all fibrils in the scaffold.

2. A composition comprising: (i) a fibrillar polymeric scaffold comprising fibrils derived from one or more polymerizable monomers, and (ii) a sacrificial polymerization scaffold, wherein the sacrificial polymerizable scaffold is adhered to the fibrillar polymeric scaffold.

3. The composition of claim 2, wherein the sacrificial polymerization scaffold comprises gelatin or poloxamer.

4. The composition of claim 3, wherein the sacrificial polymerization scaffold comprises gelatin.

5. The scaffold or composition of any one of claims 1-4, wherein the one or more polymerizable monomers comprise a fiber forming monomer.

6. The scaffold or composition of claim 5, wherein the fiber forming monomer is cotton, wool, polyester, polyamide, polyacrylonitrile, rayon, collagen, actin, fibrin, or lignin.

7. The scaffold or composition of claim 5 or 6, wherein the one or more polymerizable monomers comprises collagen.

8. The scaffold or composition of claim 7, wherein the one or more polymerizable monomers comprises type I collagen.

9. The scaffold or composition of any one of claims 1-8, wherein the one or more polymerizable monomers comprise a fibral protein.

10. The scaffold or composition of claim 9, wherein the fibral protein is zein, decorin, hyaluronic acid, cellulose, lamin, fibrinogen, fibrin, or fibronectin.

11. The scaffold or composition of claim 10, wherein the fibral protein is fibronectin.

12. The scaffold or composition of any one of claims 1-11, wherein the fibrillar polymeric scaffold is anisotropic.

13. The scaffold or composition of any one of claims 1-12, wherein the fibrillar polymeric scaffold has a thickness of at least about 300 nm.

14. The scaffold or composition of any one of claims 1-13, wherein the fibrillar polymeric scaffold comprises more than one layer.

15. The scaffold or composition of any one of claims 1-14, wherein the fibrils exhibit D-bands.

16. The scaffold or composition of any one of claims 1-15, wherein the fibrils have a substantially uniform diameter.

17. The scaffold or composition of claim 16, wherein the fibrils have a diameter of about 125 nm to about 160 nm.

18. The composition of any one of claims 2-17, wherein about 50% or greater of the fibrils are within ±20° of the orientation of all fibrils in the scaffold.

19. The scaffold or composition of claim 18, wherein about 70% or greater of the fibrils are ±20° of the orientation of all fibrils in the scaffold.

20. The scaffold or composition of claim 19, wherein about 80% or greater of the fibrils are ±20° of the orientation of all fibrils in the scaffold.

21. The scaffold or composition of anyone of claims 1-20, wherein the fibril orientation is uniform or substantially uniform throughout the fibrillar polymeric scaffold as a function of thickness.

22. A method for preparing a fibrillar polymeric scaffold comprising:
(a) contacting an adherent solid surface with a mixture comprising a polymerizable monomer and an aqueous solvent under conditions suitable to induce adhesion of polymerizable monomer to the adherent solid surface; and
(b) applying shear flow to the mixture under conditions suitable to polymerize the polymerizable monomer into fibrils,
thereby preparing the fibrillar polymeric scaffold.

23. A method for preparing a fibrillar polymeric scaffold in an apparatus, wherein the apparatus comprises two concentric cylinders, an outer cylinder that forms a reservoir having a first radius and an inner cylinder having a second radius smaller than the first radius, wherein the outer cylinder and inner cylinder are independently rotatable with respect to one another, and the outer cylinder or inner cylinder comprises an adherent solid surface in contact with the reservoir, the method comprising:
(a) providing a mixture comprising a polymerizable monomer and an aqueous solvent in the reservoir under conditions suitable to induce adhesion of polymerizable monomer to the adherent solid surface; and
(b) applying shear flow to the mixture under conditions suitable to polymerize the polymerizable monomer into fibrils,
thereby preparing the fibrillar polymeric scaffold.

24. The method of claim 23, wherein the apparatus comprises two concentric cylinders, a stationary outer cylinder that forms a reservoir having a first radius and a rotating inner cylinder having a second radius smaller than the first radius, wherein the rotating inner cylinder comprises an adherent outer solid surface.

25. The method of claim 23 or 24, wherein applying shear flow to the mixture comprises rotating the inner cylinders, the outer cylinder, or both with respect to one another to generate the shear flow.

26. The method of claim 25, wherein applying shear flow to the mixture comprises rotating the inner cylinder with respect to the outer cylinder to generate the shear flow.

27. The method of any one of claims 23-26, wherein the inner and outer cylinders are configured to move perpendicularly to one another.

28. The method of any one of claims 22-27, wherein the adherent solid surface comprises a sacrificial polymerization scaffold.

29. The method of claim 28, wherein the sacrificial polymerization scaffold comprises a gelatin or poloxamer.

30. The method of claim 29, wherein the sacrificial polymerization scaffold comprises gelatin.

31. The method of any one of claims 22-30, wherein the adherent solid surface comprises a pre-formed fibrillar polymeric scaffold.

32. The method of any one of claims 22-31, wherein the mixture comprises one or more additional fiber forming monomers.

33. The method of claim 32, wherein the one or more additional fiber forming monomers comprises a fibral protein.

34. The method of claim 33, wherein the fibral protein is zein, decorin, hyaluronic acid, cellulose, lamin, fibrinogen, fibrin, or fibronectin.

35. The method of claim 34, wherein the fibral protein is fibronectin.

36. The method of any one of claims 22-35, further comprising repeating (a) and (b) n times, wherein n is an integer from 2 to 25.

37. The method of claim 36, wherein n is 3, 4, 5, 6, 7, 8, 9, or 10.

38. The method of any one of claims 22-37, furthering comprising removing the fibrillar polymeric scaffold from the adherent solid surface.

39. The method of any one of claims 22-38, wherein the shear flow is laminar.

40. The method of any one of claims 22-39, wherein the shear flow is Couette flow.

41. The method of any one of claims 22-40, wherein the shear flow has a shear rate of from about 50 s⁻¹ to about 1000 s⁻¹.

42. The method of claim 41, wherein the shear rate is from about 50 s⁻¹ to about 100 s⁻¹.

43. The method of any one of claims 22-42, wherein the shear flow exerts a shear stress on the mixture of from about 0.5 N/m² to about 15.0 N/m².

44. The method of claim 43, wherein the shear flow exerts a shear stress on the mixture of about 1.1 N/m².

45. The method of any one of claims 22-44, wherein the mixture has a viscosity of from about 1 cP to about 25 cP.

46. A fibrillar polymeric scaffold produced according to the method of any one of claims 22-45.

47. An apparatus comprising two concentric cylinders, an outer cylinder that forms a reservoir having a first radius, an inner cylinder configured to be rotated by a motor and having a second radius smaller than the first radius, wherein the outer cylinder and inner cylinder are independently rotatable with respect to one another.

48. The apparatus of claim 47, wherein the device is configured to apply shear flow to a liquid in the reservoir.

49. The apparatus of claim 47 or claim 48, wherein the second radius is uniform along the entire length of the cylinder.

50. The apparatus of any one of claims 47-49, further comprising a motor configured to rotate the inner cylinder.

51. The apparatus of any one of claims 47-50, further comprising a base attached to the outer cylinder.

52. The apparatus of any one of claims 47-51, wherein at least one of the outer and inner cylinders is perpendicularly movable with respect to the other.

53. The apparatus of any one of claims 47-52, wherein the inner cylinder is removable from the apparatus.

54. The apparatus of any one of claims 47-53, wherein the inner cylinder and the outer cylinder are stainless steel or aluminum.

55. The apparatus of any one of claims 47-54, wherein the ratio of the diameter of the outer cylinder to the diameter of the inner cylinder is about 1.2 to 1 to about 3 to 1.

56. The apparatus of any one of claims 47-55, wherein the difference between the first radius and the second radius is from about 0.5 mm to about 3 mm.

57. The apparatus of claim 56, wherein the difference between the first radius and the second radius is about 1 mm.
